(19) United States
(12) Patent Application Publication
(10) Pub. No.: US 2005/0186146 A1
(43) Pub. Date: Aug. 25, 2005

(54) Interleukin-13 antagonist powders, spray-dried particles, and methods

(75) Inventors: David K. Gong, Belmont, CA (US); Jayne E. Hastedt, San Carlos, CA (US); John S. Patton, Palo Alto, CA (US)

Correspondence Address: NEKTAR THERAPEUTICS, 150 INDUSTRIAL ROAD, SAN CARLOS, CA, 94070, US

(73) Assignee: Nektar Therapeutics, San Carlos, CA (US)

(21) Appl. No.: 11/058,509

(22) Filed: Feb. 14, 2005

Related U.S. Application Data
(60) Provisional application No. 60/544,528, filed on Feb. 12, 2004.

(51) Int. Cl. A61K 38/17; A61L 9/04; A61K 39/395

(52) U.S. Cl. 424/46; 514/12; 424/145.1

(57) ABSTRACT

A powder includes IL-13 antagonist, wherein the powder has a mass median aerodynamic diameter (MMAD) of less than about 10 μm. A composition includes a spray-dried particle including IL-13 antagonist. A method of administering IL-13 antagonist to the lungs of a subject includes: dispersing a dry powder composition involving IL-13 antagonist to form an aerosol; and delivering the aerosol to the lungs of the subject by inhalation of the aerosol by the subject, thereby ensuring delivery of the IL-13 antagonist to the lungs of the subject. A method of treating an IL-13-related condition includes: pulmonarily administering a therapeutically effective amount of a dry powder including IL-13 antagonist. A method of preparing IL-13 antagonist-containing powder involves: combining IL-13 antagonist, optional excipient, and solvent to form a mixture or solution; and spray drying the mixture or solution to obtain the powder.

### BACKGROUND OF THE INVENTION

The present document claims priority under 35 U.S.C. § 119(e) of U.S. Provisional Application No. 60/544,528, filed Feb. 12, 2004, the disclosure of which is expressly incorporated by reference herein in its entirety.

### FIELD OF THE INVENTION

The present invention relates generally to interleukin-13 (“IL-13”) antagonists. For example, the invention relates to IL-13 antagonist-containing powders or spray-dried particles. The invention also relates to methods of administering IL-13 antagonists to the lungs. The invention further relates to methods of treating IL-13-related conditions by pulmonarily administering IL-13 antagonist. Still further, the invention relates to methods of preparing IL-13 antagonist-containing powders.

### BACKGROUND ART

Interleukin-13 (or “IL-13”) is a cytokine produced by activated T cells and has been implicated as a key factor in asthma, allergy, atopy, and inflammatory response. Specifically, IL-13 is believed to promote B-cell proliferation, induce B-cells to produce IgE, increase expression of VCAM-1 on endothelial cells, and enhance the expression of class II major histocompatibility complex antigens and various adhesion molecules on monocytes. See Moy et al. (2001) J. Mol. Biol. 310:219-230. Clinically, expression of IL-13 is implicated in airway hyperresponsiveness (or “AHR”) and inflammation, among other symptoms. Significantly, asthmatics have increased levels of IL-13 in their airways. Sypek et al. (2002) Am. J. Physiol. Lung. Cell Mol. Physiol. 282(1):L44-49. Recently, IL-13 has been shown to play a critical role in allergic asthma. Andrews et al. (2001) J. Immunol. 166(3):1716-1722.

IL-13 binds to interleukin-13 receptor (or “IL-13R”), an endogeneous protein located on the surface of certain cells. Upon binding with IL-13, IL-13R transduces a biological signal, thereby triggering a cascade of events that ultimately lead to clinical symptoms. It is known that IL-13R has several subtypes (e.g., IL-13Rα1 and IL-13Rα2) and is composed of more than one binding chain. The isolation and expression of murine IL-13 binding chains is described in U.S. Pat. No. 6,268,480.

It is believed that IL-13 will preferentially bind to soluble IL-13R (i.e., unbound IL-13R) in solution rather than to the endogenous cell-surface IL-13R, thereby preventing cellular activation and blocking of the IL-13-induced biological responses. Thus, the asthma-inducing effects of IL-13 may be reduced by the administration of exogenous IL-13R. See U.S. Pat. No. 6,248,714 and Chiaramonte et al. (1999) J. Immunol. 162(2):920-930.

Like many proteins, IL-13R is relatively instable. IL-13R tends to degrade and/or aggregate under certain conditions (e.g., highly acidic or basic pH, high temperatures) and is susceptible to oxidizing agents and endogenous proteases. The inherent chemical and physical instability of IL-13R makes pharmaceutical formulation particularly problematic. The subcutaneous administration of an agent comprising an IL-13R has been described. See U.S. Patent Application Publication 2003/0211104.

Apart from problems associated with IL-13R itself, solution-based formulations such as those typically used in subcutaneous and intravenous delivery pose their own obstacles. First, solution-based formulations take up more room and require more care than solid formulations, thereby resulting in higher costs. Moreover, in general, solution-based formulations are typically refrigerated (e.g., maintained in an environment of 2 to 8° C.), which further restricts storage and transport options. In addition, many solution-based formulations exhibit protein concentration loss over time, which is presumably due to the formation of higher order molecular aggregates in solution. Such formulations frequently must be supplemented with stabilizing additives such as buffers and/or antioxidants to minimize solution instability. Thus, it would be desirable to provide a solid or powder-based composition of IL-13R, particularly one that is both stable during preparation and storage, and administrable in solid form.

Powder formulations represent an alternative to solution formulations, and proteins, when desired in powder form, are most often prepared as lyophilizates. In the past few years, spray drying has been employed as an approach for preparing a number of therapeutic protein-based powders, particularly for aerosolized administration. See, for example, WO 96/32149, WO 95/31479, and WO 97/41833. Unfortunately, certain proteins, and cytokines in particular, are prone to degradation during spray drying, and loss of their secondary structure. See Maa et al. (1998) J. Pharm. Sciences, 87(2):152-159.

There remains, however, a need for IL-13 antagonist-containing powders and spray-dried particles. There also remains a need for methods of making and using IL-13 antagonist compositions.

### SUMMARY OF THE INVENTION

Accordingly, the present invention provides IL-13 antagonist-containing compositions, such as powders and spray-dried particles. The prevention also relates to methods of making and using IL-13 antagonist-containing compositions. Other features and advantages of the present invention will be set forth in the description of invention that follows, and in part will be apparent from the description or may be learned by practice of the invention. The invention will be realized and attained by the compositions and methods particularly pointed out in the written description and claims hereof.

A first aspect of the present invention is directed to a powder comprising IL-13 antagonist, such as a powder having a mass median aerodynamic diameter (MMAD) of less than about 10 μm.

A second aspect of the present invention is directed to a composition, comprising a spray-dried particle comprising IL-13 antagonist.

A third aspect of the present invention is directed to a method of administering IL-13 antagonist to the lungs of a subject. The method involves dispersing a composition comprising IL-13 antagonist to form an aerosol, and delivering the aerosol to the lungs of the subject by inhalation of the aerosol by the subject, thereby ensuring delivery of the IL-13 antagonist to the lungs of the subject.

A fourth aspect of the present invention is directed to a method of treating an IL-13-related condition by pulmonarily administering a therapeutically effective amount of IL-13 antagonist.

A fifth aspect of the present invention involves a method of preparing IL-13 antagonist-containing powder. The method includes combining IL-13 antagonist, optional excipient, and solvent to form a mixture or solution, and spray drying the mixture or solution to obtain the powder.

### BRIEF DESCRIPTION OF THE DRAWINGS

The present invention is further described in the description of invention that follows, in reference to the noted plurality of non-limiting drawings, wherein:

FIGS. 1A and 1B are scanning electron micrographs of two formulations according to the present invention. FIG. 1A is a scanning electron micrograph (“SEM”) of formulation A of Example 5, while FIG. 1B is an SEM of the formulation B of Example 9.

FIGS. 2A and 2B are SEM images of formulations A and B, respectively, after 1 month of storage at 40° C./75% RH in blister packs sealed in foil pouches with desiccant.

FIG. 3A shows an initial particle distribution profile for formulation A, and FIG. 4A shows the particle distribution profile for formulation A after storage in blister packs stored in foil pouches for 1 month at 40° C. and 75% relative humidity with desiccant for 1 month.

FIG. 3B shows the initial particle distribution profile for formulation B, and FIG. 4B shows the particle distribution profile for formulation B after storage in blister packs stored in foil pouches for 1 month at 40° C. and 75% relative humidity with desiccant for 1 month.

FIG. 5 shows the effect of multiple vehicle doses (comparative examples) on lung resistance in asthmatic sheep.

FIG. 6 shows the effect of increasing lung dose (mg) of vehicle (comparative examples) on lung resistance in asthmatic sheep.

FIG. 7 shows the effect of increasing lung dose (mg/kg) of vehicle (comparative examples) on lung resistance in asthmatic sheep.

FIG. 8 shows the effect of vehicle treatment (comparative examples) on the response to antigen challenge in the sheep.

FIG. 9 shows the effect of sIL-13Rα2-IgG treatment in accordance with the invention on the sheep asthmatic response.

FIG. 10A shows an initial particle distribution profile for formulation A, and FIG. 10B shows an particle distribution profile for formulation A after shipment in blister packs stored in foil pouches and desiccated.

FIG. 10C shows an initial particle distribution profile for vehicle 1 (comparative example), and FIG. 10D shows the particle distribution profile for vehicle 1 (comparative example) after shipment in blister packs stored in foil pouches and desiccated.

FIGS. 11A and 11B are SEM images of sIL-13Rα2-IgG formulations in accordance with the invention (11A) before; and (11B) after shipment in blister packs stored in foil pouches with desiccant.

FIGS. 12A and 12B are SEM images of vehicle-1 (comparative examples) formulations (12A) before; and (12B) after shipment in blister packs stored in foil pouches with desiccant.

### DESCRIPTION OF THE INVENTION

Unless otherwise stated, a reference to a compound or component includes the compound or component by itself, as well as in combination with other compounds or components, such as mixtures of compounds.

As used herein, the singular forms “a,” “an,” and “the” include the plural reference unless the context clearly dictates otherwise. Thus, for example, reference to “an IL-13R” includes a single IL-13R as well as two or more of the same or different IL-13Rs, reference to an excipient refers to a single excipient as well as two or more of the same or different excipients, and the like.

Before further discussion, a definition of the following terms will aid in the understanding of the present invention.

The term “amino acid” refers to any molecule containing both an amino group and a carboxylic acid group and can serve as an excipient. Although the amino group most commonly occurs at the beta position (i.e., the second atom from the carboxyl group, not counting the carbon of the carboxyl group) to the carboxyl function, the amino group can be positioned at any location within the molecule. The amino acid can also contain additional functional groups, such as amino, thio, carboxyl, carboxamide, imidazole, and so forth. As used herein, the term “amino acid” specifically includes amino acids as well as derivatives thereof such as, without limitation, norvaline, 2-aminoheptanoic acid, and norleucine. The amino acid may be synthetic or naturally occurring, and may be used in either its racemic or optically active (D-, or L-) forms, including various ratios of stereoisomers. The amino acid can be any combination of such compounds. Most preferred are the naturally occurring amino acids. The naturally occurring amino acids are phenylalanine, leucine, isoleucine, methionine, valine, serine, proline, threonine, alanine, tyrosine, histidine, glutamine, asparagines, lysine, aspartic acid, glutamic acid, cysteine, tryptophan, arginine, and glycine.

By “oligopeptide” is meant any polymer in which the monomers are amino acids totaling generally less than about 100 μmino acids, preferably less than 25 μmino acids. The term oligopeptide also encompasses polymers composed of two amino acids joined by a single amide bond as well as polymers composed of three amino acids.

“Dry” when referring to a powder (e.g., as in “dry powder”) is defined as containing less than about 10 wt % moisture. The compositions may have a moisture content of less than about 7 wt %, less than about 5 wt %, less than about 3 wt %, or less than about 2 wt %. The moisture of any given composition can be determined by, for example, the Karl Fischer titrimetric technique using a Mitsubishi moisture meter model # CA-06.

As used herein, an “excipient” is a non-IL13 antagonist component of a particle, powder or composition intended to be in the particle, powder, or composition. Thus, “excipients” such as buffers, sugars, amino acids, and so forth are intended components of a formulation and stand in contrast to unintended components of a formulation such as impurities (e.g., dust) and the like. Thermogravimetric analysis (“TGA”) can also be used.

A “therapeutically effective amount” is an amount of IL-13 antagonist (e.g., IL-13R) required to provide a desired therapeutic effect. The exact amount required will vary from subject to subject and will otherwise be influenced by a number of factors, as will be explained in further detail below. An appropriate “therapeutically effective amount,” however, in any individual case can be determined by one of ordinary skill in the art.

The term “substantially” refers to a system in which greater than 50% of the stated condition is satisfied. For instance, greater than 85%, greater than 92%, or greater than 96% of the condition may be satisfied.

The term “antagonist” as in “IL-13 antagonist” means a moiety that acts to diminish or eradicate the activity of IL-13. Preferred IL-13 antagonists for use with the present invention are receptors that bind to IL-13, although other moieties such as antibodies that bind to IL-13 can also be used. When administered in vivo, the exogenously administered IL-13 antagonist binds to endogenous IL-13, thereby reducing the overall amount of endogenous IL-13 available to bind to membrane-bound IL-13 receptors. In this way, there is less IL-13-initiated signal transduction, which lessens the degree of the cascade of reactions associated with, for example, the asthmatic response.

The term “IL-13R” means a poplypeptide that has the ability to bind IL-13 and includes the naturally derived or synthetically prepared animal (e.g., human, murine, and so forth) receptors IL-13R, IL-13Rα1, IL-13Rα2, a complex comprising IL-13Rα1 and IL-4α, fragments and conjugates thereof, and combinations of any of the foregoing. In addition, IL-13R includes, for example IL-13Rα2-IgG fusion protein and other immunoglobulin fusion proteins.

As used herein, “conjugate” means an IL-13 antagonist covalently bonded to another molecule. For example, conjugates include fusion proteins.

The term “subject” refers to a living organism suffering from or prone to a condition that can be prevented or treated by administration of an IL-13 antagonist (e.g., an IL-13R), and includes both humans an animals.

“Optional” and “optionally” means that the subsequently described circumstance may or may not occur, so that the description includes instances where the circumstance occurs and instances where it does not. Thus, for example, a formulation comprising an “optional excipient” includes formulations comprising one or more excipient as well as formulations lacking any excipient.

Compositions of the present invention are considered to be “respirable” if they are suitable for inhalation therapy (i.e., capable of being inspired by the mouth or nose and drawn through the airways and into the lungs) and/or pulmonary delivery (i.e., local delivery to the tissues of the deep lung and optionally absorption through the epithelial cells therein into blood circulation). Compositions of the present invention can provide for rapid action, providing, for example, therapeutically effective levels locally (e.g., at local pulmonary tissues) and/or systemically (e.g., within the systemic circulation) in less than 60 minutes. Advantageously with respect to the treatment of asthmatic systems (e.g., airway hyperreactivity, inflammation, and so on), the present compositions are effective without the need to obtain systemic circulation given that the target of the compositions is the patient's airways.

“Orally respirable” compositions are those respirable compositions that are particularly adapted for oral inhalation. Likewise, “nasally respirable” compositions are those respirable compositions that are particularly adapted for nasal inhalation, i.e., intranasal delivery into the upper respiratory tract.

“Emitted Dose” or “ED” provides an indication of the delivery of a drug formulation from a suitable inhaler device after a firing or dispersion event. More specifically, for dry powder formulations, the ED is a measure of the percentage of powder that is drawn out of a unit dose package and which exits the mouthpiece of an inhaler device. The ED is defined as the ratio of the dose delivered by an inhaler device to the nominal dose (i.e., the mass of powder per unit dose placed into a suitable inhaler device prior to firing). The ED is an experimentally determined parameter, and is typically determined using an in vitro device arranged to mimic patient dosing. To determine an ED value, a nominal dose of dry powder, typically in unit dose form, is placed into a suitable dry powder inhaler (such as described in U.S. Pat. No. 5,785,049) and then actuated, dispersing the powder. The resulting aerosol cloud is then drawn by vacuum from the device, where it is captured on a tared filter attached to the device mouthpiece. The amount of powder that reaches the filter constitutes the emitted dose. For example, for a 5 mg, dry powder-containing dosage form placed into an inhalation device, if dispersion of the powder results in the recovery of 4 mg of powder on a tared filter as described above, then the emitted dose for the dry powder composition is: 4 mg (delivered dose)/5 mg (nominal dose)×100=80%. For nonhomogenous powders, ED values provide an indication of the delivery of drug from an inhaler device after firing rather than of dry powder, and are based on amount of drug rather than on total powder weight. Similarly for MDI and nebulizer dosage forms, the ED corresponds to the percentage of drug which is drawn from a unit dosage form and which exits the mouthpiece of an inhaler device.

As used herein, a “dispersible” powder is one having an ED value of at least about 5%, such as at least about 10%, at least about 40%, at least about 55%, or at least about 70%.

“Mass median diameter” or “MMD” is a measure of mean particle size, since the powders of the invention are generally polydisperse (i.e., consist of a range of particle sizes). MMD values as reported herein are determined by centrifugal sedimentation, although any number of commonly employed techniques can be used for measuring mean particle size (e.g., electron microscopy, light scattering, laser diffraction. Typically, the MMD will be from about 0.5 micron to about 10 microns, more preferably from about 1 micron to about 5 microns.

“Mass median aerodynamic diameter” or “MMAD” is a measure of the aerodynamic size of a dispersed particle. The aerodynamic diameter is used to describe an aerosolized powder in terms of its settling behavior, and is the diameter of a unit density sphere having the same settling velocity, in air, as the particle. The aerodynamic diameter encompasses particle shape, density and physical size of a particle. As used herein, MMAD refers to the midpoint or median of the aerodynamic particle size distribution of an aerosolized powder determined by cascade impaction, unless otherwise indicated.

“Fine Particle Fraction” as in “FPF_{<3.3 μm}” or “FPF_{<4.7 μm}” is defined as the amount of particles in a powder that are under 3.3 microns or 4.7 microns, respectively, as determined by cascade impaction. With respect to FPF_{<3.3 μm}, this parameter corresponds to the total mass under stage 3 of an Anderson impactor when operated at a flow rate of 1 cfm (28.3 L/min). The actual mass of particles satisfying the stipulated size range in a given amount of powder can be calculated and is abbreviated “FPM.”

“Bulk density” refers to the density of a powder prior to compaction (i.e., the density of an uncompressed powder), and is typically measured by a well-known USP method. Typically, the compositions described herein will have a bulk density of from 0.01 to 10 grams per cubic centimeter.

“Essentially unchanged” as used in reference to the formation of higher order molecular aggregates of an IL-13 antagonist powder composition of the invention refers to a composition which exhibits a change of typically less than 5%, preferably no more than about 2% in the percentage of higher order aggregates when compared to that of the corresponding pre-dried solution or mixture.

A “minimal change” when used in reference to IL-13R monomer content in a spray dried IL-13R powder, refers to a change (i.e., decrease) in monomer content of no more than about 10% in comparison to the level of IL-13R monomer in the corresponding pre-dried solution or mixture.

“Homology” refers to the percent similarity between two polynucleotide or two polypeptide moieties. Readily available computer programs can be used to aid in the analysis of homology, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence homology are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent homology of a particular nucleotide sequence to a reference sequence can be determined using the homology algorithm of Smith and Waterman with a default scoring table and a gap penalty of six nucleotide positions.

As used herein, “fibrosis” includes any condition which involves the formation of fibrous tissue (whether such formation is desireable or undesireable). Such conditions include, without limitation, fibrositis, formation of fibromas (fibromatosis), fibrogenesis (including pulmonary fibrogenesis), fibroelastosis (including endocardial fibroelastosis), formation of fibromyomas, fibrous ankylosis, formation of fibroids, formation of fibroadenomas, formation of fibromyxomas, and fibrocystotitis (including cystic fibrosis).

As an overview, the present invention relates to IL-13 antagonist compositions and methods involving IL-13 antagonists. For instance, the present invention relates to a powder comprising IL-13 antagonist, such as a powder having a mass median aerodynamic diameter (MMAD) of less than about 10 μm.

The present invention also relates to a composition, comprising spray-dried particle comprising IL-13 antagonist.

Further, the present invention is directed to a method of administering IL-13 antagonist to the lungs of a subject. The method involves dispersing a composition comprising IL-13 antagonist to form an aerosol, and delivering the aerosol to the lungs of the subject by inhalation of the aerosol by the subject, thereby ensuring delivery of the IL-13 antagonist to the lungs of the subject.

Still further, the present invention is directed to a method of treating an IL-13-related condition by pulmonarily administering a therapeutically effective amount of IL-13 antagonist.

Yet further, the present invention involves a method of preparing IL-13 antagonist-containing powder. The method includes combining IL-13 antagonist, optional excipient, and solvent to form a mixture or solution, and spray drying the mixture or solution to obtain the powder.

Turning to exemplary aspects of the invention, the compositions include one or more IL-13 antagonist, which may take several forms. IL-13 antagonists may be antibodies, such as monoclonal antibodies. IL-13 antagonists may take the form of a soluble receptor of IL-13. Soluble receptors freely circulate in the body. When the receptor encounters IL-13, it binds to it, effectively inactivating the IL-13, since the IL-13 is then no longer able to bind with its biologic target in the body. A potent antagonist comprises two soluble receptors fused together to a specific portion of an immunoglobulin molecule (F_{c} fragment). This produces a dimer composed of two soluble receptors which have a high affinity for the target, and a prolonged half-life. Many IL-13 antagonists are known in the art. IL-13 antagonists generally have the ability to bind IL-13 with a K_{D} of about 0.1 nM to about 100 nM.

In view of the above, examples of the IL-13 antagonists include, but are not limited to, IL-13Rα1, IL-13Rα2, such as sIL-13Rα2, IL-13bc protein, IL-4/IL-13 trap, IL-13 trap, antibody to IL-13, antibody to IL-13Rα1, antibody to IL-13Rα2, antibody to IL-13bc, IL-13R-binding mutants of IL-4, small molecules capable of inhibiting the interaction of IL-13 with IL-13bc, small molecules capable of inhibiting the interaction of IL-13 with IL-13Rα1, and small molecules capable of inhibiting the interaction of IL-13 with IL-13Rα2.

Other examples of IL-13 antagonists include IL-13-binding homologs of IL-13Rα1, IL-13Rα2, such as sIL-13Rα2, IL-13bc protein, antibody to IL-13, antibody to IL-13Rα1, antibody to IL-13Rα2, and antibody to IL-13bc. The IL-13 binding homologs may have a percent homology of at least 50%, at least 60%, at least 70%, at least 80%, at least 90%, at least 95%, or at least 98%, relative to the IL-13Rα1, IL-13Rα2, such as sIL-13Rα2, IL-13bc protein, antibody to IL-13, antibody to IL-13Rα1, antibody to IL-13Rα2, or antibody to IL-13bc. For example, variants of IL-13 antagonists are disclosed in U.S. Pat. No. 5,696,234, which is incorporated by reference herein in its entirety.

Still other examples of IL-13 antagonists include binding fragments of IL-13Rα1, IL-13Rα2, such as sIL-13Rα2, IL-13bc protein, antibody to IL-13, antibody to IL-13Rα1, antibody to IL-13Rα2, and antibody to IL-13bc.

Further examples of IL-13 antagonists include conjugates, such as fusion proteins, of IL-13Rα1, IL-13Rα2, such as sIL-13Rα2, IL-13bc protein, antibody to IL-13, antibody to IL-13Rα1, antibody to IL-13Rα2, antibody to IL-13bc, homologs thereof, and IL-13-binding fragments thereof. Thus, the IL-13 antagonists may be fused to carrier molecules such as immunoglobulins. For example, soluble forms of IL-13 antagonists may be fused through “linker” sequences to the Fc portion of an immunoglobulin. IL-13 antagonists linked to immunoglobulin are disclosed in U.S. Published Application No. 2005/0235555, which is incorporated by reference herein in its entirety. Other fusion proteins, such as those with GST, Lex-A, or MBP may also be used.

Thus, conjugates include chemically modified IL-13 antagonist linked to a polymer. The polymer selected is typically water soluble so that the IL-13 antagonist to which it is attached does not precipitate in an aqueous environment, such as a physiological environment. The polymer selected is usually modified to have a single reactive group, such as an active ester for acylation or an aldehyde for alkylation, so that the degree of polymerization may be controlled as provided for in the present methods. The polymer may be of any molecular weight, and may be branched or unbranched. Included within the scope of the invention is a mixture of polymers. Preferably, for therapeutic use of the end-product preparation, the polymer will be pharmaceutically acceptable.

The polymers each may be of any molecular weight and may be branched or unbranched. The polymers each typically have an average molecular weight of between about 2 kDa to about 100 kDa (the term “about” indicating that in preparations of a water soluble polymer, some molecules will weigh more, some less, than the stated molecular weight). The average molecular weight of each polymer is typically between about 0.5 kDa and about 50 kDa, such as between about 5 kDa to about 40 kDa or between about 20 kDa to about 35 kDa.

Suitable water soluble polymers or mixtures thereof include, but are not limited to, N-linked or O-linked carbohydrates, sugars, phosphates, carbohydrates; sugars; phosphates; polyethylene glycol (PEG) (including the forms of PEG that have been used to derivatize proteins, including mono-(C1-C10) alkoxy- or aryloxy-polyethylene glycol); monomethoxy-polyethylene glycol; dextran (such as low molecular weight dextran, of, for example about 6 kD), cellulose; cellulose; other carbohydrate-based polymers, poly-(N-vinyl pyrrolidone) polyethylene glycol, propylene glycol homopolymers, a polypropylene oxide/ethylene oxide co-polymer, polyoxyethylated polyols (e.g., glycerol) and polyvinyl alcohol.

In general, chemical derivatization may be performed under any suitable condition used to react an IL-13 antagonist with an activated polymer molecule. Methods for preparing chemical derivatives of polypeptides will generally comprise (a) reacting the polypeptide with the activated polymer molecule (such as a reactive ester or aldehyde derivative of the polymer molecule) under conditions whereby the IL-13 antagonist becomes attached to one or more polymer molecules; and (b) obtaining the reaction product(s). The optimal reaction conditions will be determined based on known parameters and the desired result. For example, the larger the ratio of polymer molecules:protein, the greater the percentage of attached polymer molecule. In one embodiment, the IL-13 antagonist may have a single polymer molecule moiety at the amino terminus. (See, e.g., U.S. Pat. No. 5,234,784).

A particularly preferred water-soluble polymer for use herein is polyethylene glycol, abbreviated PEG. As used herein, polyethylene glycol is meant to encompass any of the forms of PEG that have been used to derivatize other proteins, such as mono-(C1-C10) alkoxy- or aryloxy-polyethylene glycol. PEG is a linear or branched neutral polyether, available in a broad range of molecular weights, and is soluble in water and most organic solvants. PEG is effective at excluding other polymers or peptides when present in water, primarily through its high dynamic chain mobility and hydrophibic nature, thus creating a water shell or hydration sphere when attached to other proteins or polymer surfaces. PEG is nontoxic, non-immunogenic, and approved by the Food and Drug Administration for internal consumption.

Proteins or enzymes when conjugated to PEG have demonstrated bioactivity, non-antigenic properties, and decreased clearance rates when administered in animals. F. M. Veronese et al., Preparation and Properties of Moonomthoxypoly(ethylene glyco.)-modified Enzymes for Therapeutic Applications, in J. M. Harris ed., Poly(Ethylene Clycol) Chemistry-Biotechnical and Biomedical Applications 127-36, 1992, incorporated herein by reference. This is due to the exclusion properties of PEG in preventing recognition by the immune system. In addition, PEG has been widely used in surface modification procedures to decrease protein adsorption and improve blood compatibility. S. W. Kim et al, Ann. N. Y Acad. Sci. 516: 116-30 1987; Jacobs et al., Artif. Organs 12: 500-501, 1988; Park et al., J. Poly. Sci, Part A 29:1725-31, 1991, incorporated herein by reference. Hydrophobic polymer surfaces, such as polyurethanes and polystyrene were modified by the grafting of PEG (MW 3400) and employed as nonthrombogenic surfaces. In these studies, surface properties (contact angle) were more consistent with hydrophilic surfaces, due to the hydrating effect of PEG. More importantly, protein (albumin and other plasma proteins) adsorption was greatly reduced, resulting from the high chain motility, hydration sphere, and protein exclusion properties of PEG.

PEG (MW 3400) was determined as an optimal size in surface immobilization studies, Park et al., J. Biomed. Mat. Res. 26:739-45, 1992, while PEG (MW 5000) was most beneficial in decreasing protein antigenicity. (F. M. Veronese et al., In J. M. Harris et., Poly(Ethylene Glycol) Chemistry—Biotechnical and Biomedical Applications 127-36, supra., incorporated herein by reference).

In general, chemical derivatization may be performed under any suitable conditions used to react a biologically active substance with an activated polymer molecule. Methods for preparing pegylated IL-13 antagonist will generally comprise (a) reacting the IL-13 antagonist with polyethylene glycol (such as a reactive ester or aldehyde derivative of PEG) under conditions whereby the IL-13 antagonist becomes attached to one or more PEG groups; and (b) obtaining the reaction product(s). In general, the optimal reaction conditions for the acylation reactions will be determined based on known parameters and the desired result. For example, the larger the ratio of PEG:protein, the greater the percentage of poly-pegylated product.

IL-13R for use in the compositions described herein may be purchased from a commercial source or may be recombinantly produced, for example, using a process described in Miloux et al. (1997) FEBS letter 401(2-3): 163-166 or Zhang et al. (1997) J. Biol Chem 272:16921-16926. With resepect to IL-13Rα1, for example, the coding region is 1284 base pairs long including a stop codon at the 3′ terminus. Cloning and characterization of murine IL-13Rα1 has been described. See Hilton et al. (1996) Proc. Natl. Acad. Sci. USA 93:497-501. With respect to human IL13Rα1, the protein is believed to consist of 427 amino acid residues and has also been cloned and characterized. See Aman et al. (1996) J. Biol. Chem. 271(46) 29265-292670. A preferred receptor is comprised of paired IL-13Rα1 and IL-4Rα and has been found to bind IL-13 particularly well. See Andrews et al. (2001) J. Immunol. 166(3):1716-1722. Those of ordinary skill in the art can prepare recombinant versions of IL-13R based on the references cited herein or elsewhere in the literature. In addition, naturally occurring IL-13R can be obtained by lysing cells and recovering the membrane bound IL-13R by known separation techniques such as centrifugation and chromatography.

IL-13bc, homologs thereof, fragments thereof, and conjugates thereof are disclosed in U.S. Pat. No. 6,664,227, which is incorporated by reference herein in its entirety.

The IL-13 antagonist may be neutral (i.e., uncharged) or may be in the form of a pharmaceutically acceptable salt, for example, an acid addition salt such as acetate, maleate, tartrate, methanesulfonate, benzenesulfonate, toluenesulfonate, and so forth, or an inorganic acid salt such as hydrochloride, hydrobromide, sulfate, phosphate, and so on. Cationic salts may also be employed, such as salts of sodium, potassium, calcium, magnesium, or ammonium salts. Regardless of whether the IL-13 antagonist is charged, uncharged, or in a salt form, the IL-13 antagonists are preferably soluble upon administration to a patient. That is, at least some fraction of the total IL-13R solubilizes in vivo in order to effect binding of endogenous IL-13.

The IL-13 antagonist-containing compositions of the present invention may take various forms. For instance, the composition may be in the form of a powder, spray-dried particles, or a solution for nebulization.

The amount of IL-13 antagonist contained within the composition may be sufficient to pulmonarily deliver a therapeutically effective amount (i.e., amount required to exert the therapeutic effect) of IL-13 antagonist per unit dose over the course of a dosing regimen. In practice, this will vary depending upon the particular IL-13 antagonist (e.g., natural vs. synthetic, full-length vs. fragment and its corresponding bioactivity), the patient population, and dosing requirements. Due to the highly dispersible nature of some of the respirable powders of the invention, losses to the inhalation device are minimized, meaning that more of the powder dose is actually delivered to the patient. This, in turn, correlates to a lower required dosage to achieve the desired therapeutic goal.

In general, the total amount of IL-13 antagonist contained in the compositions will range from about 1 wt % to 100 wt %, based on the total weight of the composition, such as from about 2 wt % to 100 wt %, about 5 wt % to about 98%, (e.g., about 5 wt % to 60 wt %), about 10 wt % to about 95 wt %, about 45 wt % to about 95 wt %, or about 50 wt % to about 90 wt %. For instance, a dry powder composition may contain IL-13R in an amount ranging from about 40 wt % to about 80 wt % or in an amount ranging from about 0.2 wt % to about 99 wt %.

The actual therapeutically effective amount of IL-13 antagonist will vary from one patient to the next and from one therapeutic regimen to the next. The amount and frequency of administration will depend, of course, on factors such as the nature and severity of the indication being treated, the desired response, the patient population, condition of the patient, and so forth. Generally, a therapeutically effective amount will range from about 0.001 mg/kg/dose to 100 mg/kg/dose, such as from 0.01 mg/kg/dose to 75 mg/kg/dose, or from 0.10 mg/kg/day to 50 mg/kg/dose.

Each dose can be administered in a variety of dosing schedules, again depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Once the clinical endpoint has been achieved, dosing is halted.

The composition of the invention may also contain one or more additional active ingredient. Examples of other active ingredients include, but are not limited to, cytokines (e.g., immune modulating cytokine), cytokine antagonists (e.g., IL-4 antagonist), lymphokines, or other hematopoietic factors such as M-CSF, GM-CSF, interleukins (such as, IL-1, IL-2, IL-3, IL-4. IL-24, IL-25), G-CSF, stem cell factor, and erythropoietin. The composition may also include anti-cytokine antibodies. The composition may further contain other anti-inflammatory agents. Such additional factors and/or agents may be included in the composition to produce a synergistic effect with isolated IL-13 antagonist, or to minimize side effects caused by the isolated IL-13 antagonist. Conversely, IL-13 antagonist may be included in formulations of the particular cytokine, lymphokine, other hematopoietic factor, thrombolytic or anti-thrombotic factor, or anti-inflammatory agent to minimize side effects of the cytokine, lymphokine, other hematopoietic factor, thrombolytic or anti-thrombotic factor, or anti-inflammatory agent.

In view of the above, examples of other active ingredients include, but are not limited to, one or more of inhaled asthma medication, such as but not limited to an asthma related therapeutic, a TNF antagonist, an antirheumatic, a muscle relaxant, a narcotic, an analgesic, an anesthetic, a sedative, a local anethetic, a neuromuscular blocker, an antimicrobial, an antipsoriatic, a corticosteriod, an anabolic steroid, an asthma related agent, a mineral, a nutritional, a thyroid agent, a vitamin, a calcium related hormone, an antidiarrheal, an antitussive, an antiemetic, an antiulcer, a laxative, an anticoagulant, an erythropieitin, a filgrastim, a sargramostim, an immunization, an immunoglobulin, an immunosuppressive, a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha, a cytokine, a cytokine antagonist.

In particular, asthma related compositions of the invention can optionally further comprise at least one selected from an asthma-related therapeutic, a TNF antagonist (e.g., but not limited to a TNF Ig derived protein or fragment, a soluble TNF receptor or fragment, fusion proteins thereof, or a small molecule TNF antagonist), an antirheumatic, a muscle relaxant, a narcotic, a non-steroid anti-inflammatory drug (NSAID), an analgesic, an anesthetic, a sedative, a local anethetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, an asthma related agent, a mineral, a nutritional, a thyroid agent, a vitamin, a calcium related hormone, an antidiarrheal, an antitussive, an antiemetic, an antiulcer, a laxative, an anticoagulant, an erythropieitin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonistm. Suitable amounts and dosages are well known in the art. See, e.g., Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

The compositions of the present invention may be formulated “neat,” i.e. without pharmaceutical excipients or additives. In addition, the compositions can also be prepared to optionally include one or more pharmaceutically acceptable excipients. Such excipients, if present, are generally present in the powder composition in amounts ranging from about 0.01 wt % to about 99 wt %, about 0.1 wt % to about 95 wt %, about 0.5 wt % to about 80 wt %, or about 1 wt % to about 60 wt %. The Examples section describes various excipient-containing IL-13 antagonist compositions. Typically, the excipient or excipients will serve to improve one or more of the following: the aerosol properties of the composition; chemical stability; physical stability; storage stability; and handling characteristics.

In particular, the excipient materials can often function to improve the physical and chemical stability of the IL-13 antagonist compositions. For example, the excipient may minimize the residual moisture content and hinder moisture uptake and/or enhance particle size, degree of aggregation, surface properties (i.e., rugosity), ease of inhalation, and targeting of the resultant particles to the lung. The excipient(s) may also simply serve simply as bulking agents for reducing the active agent concentration in the dry powder composition.

Pharmaceutical excipients useful in the present composition include, but are not limited to, proteins (i.e., large molecules composed of one or more chains of amino acids in a specific order), oligopeptides (i.e., short chains of amino acids connected by peptide bonds), peptides (i.e., a class of molecules that hydrolyze into amino acids), amino acids, lipids (i.e., fatty, waxy or oily compounds typically insoluble in water but soluble in organic solvents, containing carbon, hydrogen and, to a lesser extent, oxygen), polymers, and carbohydrates (e.g., sugars, including monosaccharides, di-, tri-, tetra-, and oligosaccharides; derivatized sugars such as alditols, aldonic acids, esterfied sugars and the like; and polysaccharides or sugar polymers), which may be present singly or in combination. Suitable excipients include those provided in International Publication No. WO 96/32096.

Preferred excipients include sugar alcohols, lipids, DPPC, DSPC, calcium/magnesium, amino acids (particularly hydrophobic amino acids), oligopeptides, polypeptides, and sugars (particularly hydrophobic sugars). Particularly preferred excipients include zinc salts, leucine, citrate, and sugars such as sucrose and mannitol. For particulate formulations, preferred excipients are those having glass transition temperatures (Tg), above about 35° C., such as above about 45° C., or above about 55° C.

Exemplary polypeptide and protein excipients include serum albumin such as human serum albumin (HSA), recombinant human albumin (rHA), gelatin, casein, hemoglobin, and the like. For instance, dispersibility enhancing polypeptides, e.g., HSA, as described in international Publication No. WO 96/32096, may be used.

Representative amino acid/polypeptide components, which may also function in a buffering capacity, include alanine, glycine, arginine, betaine, histidine, glutamic acid, aspartic acid, cysteine, lysine, leucine, isoleucine, valine, methionine, phenylalanine, aspartame, tyrosine, tryptophan, and the like. Preferred are amino acids and peptide that function as dispersing agents. Amino acids falling into this categoray include hydrophobic amino acids such as leucine (leu), valine (val), isoleucine (isoleu), tryptophan (try) alinine (ala), methionine (met), phenylalanine (phe), tyrosine (try), histidin (his), and proline (pro). One particularly preferred amino acid is the amino acid leucine. Leucine, when use in the formulations described herein, includes D-leucine, L-leucine, and racemic leucine. Dispersibility enhancing peptides for use in the invention include dimers, trimers, tetramers, and pentamers composed of hydrophobic amino acid components such as those described above. Examples include di-leucine, di-valine, di-isoleucine, di-tryptophan, di-alanine, and the like, tripleucine, tripvaline, tripisoleucine, triptryptophan etc.; mixed di- and tri-peptides, such as leu-val, isoleu-leu, try-ala, leu-try, etc., and leu-val-leu, val-isoleu-try, ala-leu-val, and the like and homo-tetramers or pentamers such as tetra-alanine and penta-alanine. Particularly preferred oligopeptide excipients are dimers and trimers composed of 2 or more leucine residues, as described in International Patent Application PCT/US00/09785. Thus for example, preferred oligopeptides are selected from the group consisting of dileucine, leu-leu-gly, leu-leu-ala, leu-leu-val, leu-leu-leu, leu-leu-ile, leu-leu-met, leu-leu-pro, leu-leu-phe, leu-leu-trp, leu-leu-ser, leu-leu-thr, leu-leu-cys, leu-leu-tyr, leu-leu-asp, leu-leu-glu, leu-leu-lys, leu-leu-arg, leu-leu-his, leu-leu-nor, leu-gly-leu, leu-ala-leu, leu-val-leu, leu-ile-leu, leu-met-leu, leu-pro-leu, leu-phe-leu, leu-trp-leu, leu-ser-leu, leu-thr-leu, leu-cys-leu, leu-try-leu, leu-asp-leu, leu-glu-leu, leu-lys-leu, leu-arg-leu, leu-his-leu, leu-nor-leu, and combinations thereof. Of these, dileucine and trileucine are particularly preferred.

Another preferred feature of an excipient for use in the invention is surface activity. Surface active excipients, which may also function as dispersing agents, such as hydrophobic amino acids (e.g., leu, val isoleu, phe, etc.), di- and tri-peptides, polyamino acids (e.g., polyglutamic acid) and proteins (e.g., HSA, rHA, hemoglobin gelatin) are particularly preferred, since due to their surface active nature, these excipients tend to concentrate on the surface of the particles of the IL-13 antagonist composition, making the resultant particles highly dispersible in nature. Other exemplary surface active agents that may be included in the IL-13 antagonist compositions described herein include but are not limited to polysorbates, lecithin, oleic acid, benzalkonium chloride, and sorbitan esters.

Carbohydrate excipients suitable for use in the invention include, for example; monosaccharides such as fructose, maltose, galactose, glucose, d-mannose, sorbose, and the like; disaccharides, such as sucrose, raffinose, melezitose, maltodestrins, dextrans, straches and the like; and alditols, such as mannitol, xylitol, maltitol, lactitol, xylitol sorbital (glucito), myoinasitol and the like.

The IL-13 antagonist compositions may also include a buffer or a pH-adjusting agent; typically, the buffer is a salt prepared from an organic acid or base. Representative buffers include organic acid salts such as salts of citric acid (to provide the corresponding citrate), ascorbic acid, gluconic acid, carbonic acid, taratric acid, succinic acid, acetic acid, or phthalic acid, Tris, tromethamine hydrochloride, or phosphate buffer. In one or more embodiments, sufficient buffer, e.g., a citrate, is included to minimize degradation of the IL-13 antagonist, and the amount of buffer does not have a negative effect on lung resistance. For instance, the composition may include less than about 20 wt % of the buffer, such as less than about 10 wt %, less than about 8 wt %, less than about 5 wt %, or less than about 3 wt %. In one or more embodiments, the amount of buffer is less than about 20 mg, such as less than about 15 mg, less than about 10 mg, or less than about 5 mg. Similarly, in one or more embodiments, the amount of buffer is less than about 1 mg/kg, such as less than about 0.8 mg/kg, less than about 0.6 mg/kg, less than about 0.4 mg/kg, or less than about 0.2 mg/kg.

Additionally, the IL-13 antagonist compositions of the invention may include polymeric excipients/additives such as polyvinylpyrrolidones, derivatized celluloses such as hydroxypropylmethylcellulose, Ficcols (a polyeric sugar), hydroxyethylsartch, dextrates (e.g., cyclodextrins, such as 2-hydroxypropyl-β-cyclodextrin and sulfobutylether-β-cyclodextrin), polyethylene glycols, salts (e.g., sodium chloride), antimicrobial agents, antioxidants, antistatic agents, surfactants (e.g., polysorbates such as “TWEEN 20” and “TWEEN 80”), lecithin, oleic acid, benzalkonium chloride, sorbitan esters, lipids (e.g., phospholipids, fatty acids), steroids (e.g., cholesterol) and chelating agents (e.g., EDTA). For compositions containing a polymeric component, the polymer may typically be present to a limited extent in the composition, i.e., at levels less than about 10% by weight. Preferred compositions of the invention are those in which the IL-13 antagonist is nonliposomally or polymer encapsulated, or noncoated (i.e., absent a discrete coating layer). Preferred IL-13 antagonist compositions such as those exemplified herein are immediate-acting formulations, i.e., designed for immediate rather than for sustained release applications.

Other pharmaceutical excipients and/or additives suitable for use in the IL-13 antagonist compositions according to the invention are listed in “Remington: the Science & Practice of Pharmacy,” 19^{th} ed., Williams & Williams, (1995), in the “Physician's Desk Reference,” 52^{nd} ed., Medical Economics, Montvale, N.J. (1998), and in “The Handbook of Pharmaceutical Excipients,” 3^{rd} Edition, A. H. Kibbe, ed., American Pharmaceutical Association, Pharmaceutical Press, 2000.

In accordance with the invention, the IL-13 antagonist compositions may be a dry powder, the dry powder being crystalline, an amorphous glass, or a mixture of both forms. For formulations containing a surface-active agent, the surface-active material (in either crystalline or amorphous form), will typically be present on the surface of the particles in a higher concentration than in the bulk powder.

The compounds, powders, and spray-dried particles of the present invention may be made by any of the various methods and techniques known and available to those skilled in the art.

For example, IL-13 antagonist-containing powder compositions, such as dry powder formulations may be prepared by spray drying. Spray drying is carried out, for example, as described generally in the Spray-drying Handbook,” 5^{th} ed., K. Masters, John Wiley & Sons, Inc., NY, N.Y. (1991), and in Platz, R., et al., International Patent Publication Nos. WO 97/41833 (1997) and WO 96/32149 (1996).

Briefly, to prepare an IL-13 antagonist-containing solution for spray drying, IL-13 antagonist (and any other excipients) is generally dissolved or mixed in water, optionally containing a physiologically acceptable buffer. The pH range of solution is generally between about 3 and 10, with nearer neutral pHs being preferred, since such pHs may aid in maintaining the physiological compatibility of the powder after dissolution of powder within the lung. The aqueous formulation may optionally contain additional water-miscible solvents, such as acetone, alcohols and the like. Representative alcohols are lower alcohols such as methanol, ethanol, propanol, isopropanol, and the like. The solutions will generally contain IL-13 antagonist dissolved at a concentration from about 0.01% (w/v) to about 20% (w/v), such as from about 0.1% to about 10% (w/v), or from about 1% (w/v) to about 3% (w/v). Alternatively, components of the IL-13 antagonist formulation may be spray dried using an organic solvent or co-solvent system, employing one or more solvents such as acetone, alcohols (e.g., methanol and ethanol), ethers, aldehydes, hydrocarbons, ketones and polar aprotic solvents.

The IL-13 antagonist-containing solutions may be spray dried in a known spray drier, such as those available from commercial suppliers such as Niro A/S (Denmark), Buchi (Switzerland) and the like, resulting in a dispersible, respirable IL-13 antagonist composition, preferably in the form of a respirable dry powder. Optimal conditions for spray-drying the active agent solutions will vary depending upon the formulation components, and are generally determined experimentally. The gas used to spray-dry the material is typically air, although inert gases such as nitrogen or argon are also suitable. Moreover, the temperature of both the inlet and outlet of the gas used to dry the sprayed material is such that it does not cause decomposition of the IL-13 antagonist in the sprayed material. Such temperatures are typically determined experimentally, although generally, the inlet temperature will range from about 50° C. to about 200° C. while the outlet temperature will range from about 30° C. to about 150° C.

Alternatively, the IL-13 antagonist powder compositions may be prepared by lyophilization, vacuum drying, spray freeze drying, super critical fluid processing, air drying, or other forms of evaporative drying. Milling and other particle-size reduction techniques can also be used to provide particles.

In some instances, it may be desirable to provide the IL-13 antagonist powder formulation in a form that possesses improved handling/processing characteristics, e.g., reduced static, better flowability, low caking and the like, by preparing compositions composed of fine particle aggregates, that is, aggregates or agglomerates of the above-described respirable IL-13R. Dry powder particles, where the aggregates are readily broken back down to the fine powder components for pulmonary delivery, as described in, e.g., U.S. Pat. No. 5,654,007. Alternatively, the IL-13 antagonist powders may be prepared by agglomerating the powder components, sieving the materials to obtain the agglomerates, spheronizing to provide a more spherical agglomerate, and sizing to obtain a uniformly-sized product, as described in, e.g., International PCT Publication No. WO 95/09616.

The IL-13 antagonist powders are preferably maintained under dry (i.e., relatively low humidity) conditions during manufacture, processing, and storage. Irrespective of the drying process employed, the process will preferably result in respirable, highly dispersible compositions composed of substantially amorphous IL-13R particles.

Certain physical characteristics of the spray dried IL-13 antagonist compositions are preferred to maximize the efficiency of aerosolized delivery of such compositions to the lung.

The IL-13 antagonist compositions may be composed of particles effective to penetrate into the lungs. Passage of the particles into the lung physiology is an important aspect of the present invention. To this end, the particles of the invention have a mass median diameter (MMD) of less than about 10 μm, such as less than about 7.5 μm, less than about 5 μm. The MMD usually ranges from about 0.1 μm to about 5 μm, such as about 0.5 to 3.5 μm. The IL-13 antagonist compositions may also contain non-respirable carrier particles such as lactose, where the non-respirable particles are typically greater than about 40 microns in size. In a preferred embodiment, the dry powder is non-liposomal or non-lipid containing.

The IL-13 antagonist compositions of the invention may have an aerosol particle size distribution less than about 10 μm mass median aerodynamic diameter (MMAD), such as less than about 5 μm, or less than about 3.5 μm. The MMAD will characteristically range from about 0.5 μm to about 10 μm, such as about 0.5 μm to about 5 μm, about 0.5 μm to about 4 μm, about 1 μm to about 4 μm, about 1 μm to about 3.5 μm, or about 1.5 μm to about 2.5 μm.

The IL-13 antagonist compositions of the invention can have an emitted dose of greater than about 60%, such as greater than about 65%, greater than about 70%, greater than about 75%, or greater than about 80%.

The IL-13 antagonist compositions, particularly the respirable dry powder compositions, generally have a moisture content below about 10 wt %, such as below about 5 wt % or below about 3 wt %. Such low moisture-containing solids tend to exhibit a greater stability upon packaging and storage.

The dry powders preferably have a bulk density ranging from about 0.1-10 g/cc, such as about 0.25-4 g/cc, about 0.5-2 g/cc, or about 0.7-1.4 g/cc.

An additional measure for characterizing the overall aerosol performance of a dry powder is the fine particle dose or mass (FPM) or fine particle fraction (FPF), which describes the mass percentage of powder having an aerodynamic diameter less than a certain amount (e.g., 3.3 microns or 4.7 microns). Dry powders may have an FPF value greater than 40% (or 0.40), such as greater than 50% (or 0.50), greater than 60% (0.60), or greater than 70% (0.70), or range from about 0.4 to about 0.95, or from about 0.5 to about 7. Powders containing at least fifty percent of aerosol particles sized between about 0.5 μm and about 3.5 μm are extremely effective when delivered in aerosolized form, in reaching the regions of the lung, including the alveoli.

The spray-dried IL-13 antagonist-containing powder compositions of the present invention preferably have an essentially unchanged higher order molecular aggregate as compared to that of its pre-spray-dried solution or mixture. In other words, the spray drying process does not induce the formation of linked molecular species or other aggregates, thereby affecting the overall percent of the amount of higher order molecular aggregates in the composition. That is to say, the change in higher order molecular aggregates between spray dried powder and pre-spray dried solution or suspension is “essentially unchanged,” e.g., the percentage of monomer content of spray dried powder as compared to that of the pre-spray-dried solution or suspension is typically no more than about 15%, such as no more than about 10%, no more than about 7%, or about 5% or less.

The IL-13 antagonist powder compositions of the present invention are typically “storage stable,” i.e., characterized by minimal molecular aggregate formation and/or minimal particulate aggregate formation, when stored for extended periods at extreme temperatures (“temperature stable”) and humidities (“moisture stable”). For example, the spray dried IL-13 antagonist compositions of the present invention experience minimal particulate aggregate formation and minimal formation of higher order molecular aggregates after storage for a period of time (e.g., two weeks or more) at a temperature ranging from about 2° C. to about 50° C., such as about 25° C., and/or a relative humidity (“RH”) ranging from 0% to about 75%, such as about 33% RH. Specifically, the stored IL-13 antagonist-containing powder compositions of the present invention preferably form less than about 15% insoluble aggregates (as compared to the pre-spray-dried solutions or mixtures), such as less than about 10% insoluble aggregates, less than about 7% insoluble aggregates, less than about 5% insoluble aggregates, less than about 2.5% insoluble aggregates, less than about 2% insoluble aggregates, or less than about 1% insoluble aggregates. Alternatively, the stored IL-13 antagonist-containing powder compositions of the present invention preferably experience an increase in higher order molecular aggregate content that is no more than about 20%, such as no more than about 10%, no more than about 7%, or less than about 5%, less than about 2.5%, less than about 2%, or less than about 1%.

The IL-13 antagonist powders and particles of the present invention may be highly dispersible and respirable. Thus, the present powders and particles may be delivered pulmonarily or intranasally. The powder compositions described herein overcome many of the problems often encountered heretofore in administering peptide agents, particularly the problems associated with solution-based formulations of IL-13 antagonists. Examples of such problems include prolonged response time (e.g., time between administration and onset of physiological response), low systemic absorption and relatively low concentrations in tissues and secretions, the inability to maintain acceptable local or serum levels, and the instability of peptides, and cytokines in particular, in solution.

The present invention also includes formulations for nebulization. Formulations for nebulization are generally known in the art. Respirable powder-based formulations and nebulized formulations are distinct. Despite the fact that nebulized formulations may be considered by some to be “inhaleable,” in that they are breathed through the mouth and into the lungs, they are not “respirable” as defined herein. For example, nebulized formulations typically cannot reach the tissues of the deep lung. Moreover nebulized formulations are solution-based, i.e., are administered in solution rather than in solid form.

The compositions of the present invention may be used to treat IL-13-related conditions. Examples of IL-13-related conditions include, but are not limited to, inflammation; fibrosis (such as idiopathic pulmonary fibrosis, bleomycin-induced pulmonary fibrosis, radiation-induced pulmonary fibrosis, pulmonary granuloma, and hepatic fibrosis); chronic graft rejection; progressive systemic sclerosis; schistosomiasis; Ig-mediated conditions and diseases, particularly IgE-mediated conditions (including without limitation atopy, allergic conditions, asthma, immune complex diseases (such as, for example, lupus, nephrotic syndrome, nephritis, glomerulonephritis, thyroiditis and Grave's disease)); immune deficiencies, specifically deficiencies in hematopoietic progenitor cells, or disorders relating thereto; cancer and other disease. Such pathological states may result from disease, exposure to radiation or drugs, and include, for example, leukopenia, bacterial and viral infections, anemia, B cell or T cell deficiencies such as immune cell or hematopoietic cell deficiency following a bone marrow transplantation. Since IL-13 inhibits macrophage activation, IL-13 antagonists may also be useful to enhance macrophage activation (i.e., in vaccination, treatment of mycobacterial or intracellular organisms, or parasitic infections). IL-13 antagonists may also be useful in treating HIV infection and AIDS.

The IL-13 antagonist compositions of the present invention are particularly effective for the treatment of allergic diseases and conditions, such as asthma. Thus, the present invention also provides a method for modulating or treating asthma related conditions, in a cell, tissue, organ, or patient (human or animal) including, but not limited to, at least one of asthma, bronchial inflammation, excess bronchial mucus or plugs, lung tissue damage, eosinophil accumulation, bronchospasm, narrowing of breathing airways, airway hypersensitivity, airway remodeling, associated pulmonary or sinus inflammation leading to at least one of inspatory or expiatory airway, wheezing, breathlessness, chest tightness, coughing, dyspnea, burning, airway edema, excess mucus, bronchospasm, tachypnea, tachycardia, cyanosis, allergic rhinitis, infections (e.g., fungal or bacterial), allergy; atopic dermatitis; biorhythm abnormalities; Churg-Strauss syndrome; flu vaccination; gastroesophageal reflux disease; hay fever; indoor allergies, and the like. Such a method can optionally comprise administering an effective amount of at least one composition or pharmaceutical composition comprising at least one asthma related Ig derived protein to a cell, tissue, organ, animal or patient in need of such modulation, treatment or therapy.

The present invention also provides a method for modulating or treating at least one asthma associated immune related disease, in a cell, tissue, organ, animal, or patient including, but not limited to, at least one of asthma, associated pulmonary or sinus inflammation leading to at least one of inspatory or expatory wheezing, breathlessness, chest tightness, coughing, dyspnea, burning, airway edema, excess mucus, bronchospasm, tachypnea, tachycardia, cyanosis, allergic rhinitis, infections (e.g., fungal or bacterial), and the like. See, e.g., the Merck Manual, 12th-17th Editions, Merck & Company, Rahway, N.J. (1972, 1977, 1982, 1987, 1992, 1999), Pharmacotherapy Handbook, Wells et al., eds., Second Edition, Appleton and Lange, Stamford, Conn. (1998, 2001), each entirely incorporated by reference.

Any method of the present invention can comprise administering an effective amount of a composition or pharmaceutical composition comprising at least one IL-13 antagonist to a cell, tissue, organ, animal or patient in need of such modulation, treatment or therapy. Such a method can optionally further comprise co-administration or combination therapy for treating such asthma related diseases, wherein the administering of the IL-13 antagonist, further comprises administering, before concurrently, and/or after, at least one asthma-related therapeutic, a TNF antagonist (e.g., but not limited to a TNF Ig derived protein or fragment, a soluble TNF receptor or fragment, fusion proteins thereof, or a small molecule TNF antagonist), an antirheumatic, a muscle relaxant, a narcotic, a non-steroid anti-inflammatory drug (NSAID), an analgesic, an anesthetic, a sedative, a local anesthetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, an asthma related agent, a mineral, a nutritional, a thyroid agent, a vitamin, a calcium related hormone, an antidiarrheal, an antitussive, an antiemetic, an antiulcer, a laxative, an anticoagulant, an erythropieitin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonistm. Suitable dosages are well known in the art. See, e.g., Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

The IL-13 antagonist-containing powder compositions, particularly the dry powder compositions described herein, are preferably delivered using any suitable dry powder inhaler (DPI), i.e., an inhaler device that utilizes the patient's inhaled breath as a vehicle to transport the previously dispersed (by passive or active means) dry powder to the lungs. Preferred dry powder inhalation devices described U.S. Pat. Nos. 5,458,135, 5,740,794, and 5,785,049, and in International Patent Publication WO 00/18084.

When administered using a device of this type, the IL-13 antagonist composition is contained in a receptacle having a puncturable lid or other access surface, preferably a blister package or cartridge, where the receptacle may contain a single dosage unit or multiple dosage units. Large numbers of cavities are conveniently filled with metered doses of dry powder medicament as described in International Patent Publication WO 97/41031.

Also suitable for delivering the IL-13 antagonist compositions described herein are dry powder inhalers of the type described in, for example, U.S. Pat. No. 3,906,950 and 4,013,075, wherein a pre-measured dose of dry powder for delivery to a subject is contained within a hard gelatin capsule.

Other dry powder dispersion devices for pulmonary administration of dry powders include those described in, for example, European Patent Nos. EP 129985, EP 472598, EP 467172, and in U.S. Pat. No. 5,522,385. Also suitable for delivering the IL-13R powder compositions of the invention are inhalation devices such as the Astra-Draco “TURBUHALER.” This type of device is described in detail in U.S. Pat. Nos. 4,668,218; 4,667,668; and 4,805,811. Also suitable are devices which employ the use of a piston to provide air for either entraining powdered medicament, lifting medicament from a carrier screen by passing air through the screen, or mixing air with powder medicament in a mixing chamber with subsequent introduction of the powder to the patient through the mouthpiece of the device, such as described in U.S. Pat. No. 5,388,572.

The inhaleable IL-13 antagonist compositions may also be delivered using a pressurized, metered dose inhaler (MDI) containing solution or suspension of drug, e.g., dry powder, in a pharmaceutically inert liquid propellant, e.g., a chlorofluorocarbon or fluorocarbon, as described in U.S. Pat. Nos. 5,320,094 and 5,672,581. Prior to use, the IL-13 antagonist compositions are generally stored in a receptacle under ambient conditions, and preferably are stored at temperatures at or below about 25° C., and relative humidities ranging from about 30 to 60%. More preferred relative humidity conditions, e.g., less than about 30%, may be achieved by the incorporation of desiccating agent in the secondary packaging of the dosage form. The respirable dry powders of the invention are characterized not only by good aerosol performance, but by good stability, as well.

When aerosolized for direct delivery to the lung, the IL-13 antagonist compositions described herein will exhibit good in-lung bioavailabilities.

Asthma related therapies that can optionally be combined with at least one IL-13 antagonist for methods or compositions of the present invention, include any medication or treatment that can be used to treat an asthma related condition, disease, symptom or the like. Specific non-limiting examples of asthma therapies that are optionally included in methods of the present invention include, beta-2 agonists, anticholinergics, corticosteroids, glucocorticosteroids, anti-allergenics, anti-inflammatories, bronchiodialators, expectorants, allergy medications, cromolyn sodium, albuterol, Ventolin™, Proventil™; beclomethasone dipropionate inhaler, Vanceril™; budesonide inhaler, Pulmicort Turbuhaler™, Pulmicort Respules™; fluticasone and salmeterol oral inhaler, Advair™ Diskus; fluticasone propionate oral inhaler, Flovent™; hydrocortisone oral, Hydrocortone™, Cortef™; ipratropium bromide inhaler, Atrovent™; montelukast, Singulair™; prednisone, Deltasone™, Liquid Pred™; salmeterol, Serevent™; terbutaline, Brethine™; Bricanyl™; theophylline, Theo-Dur™, Respbid™, Slo-Bid™, Theo-24™, Theolair™, Uniphyl™, Slo-Phyllin™; triamcinolone acetonide inhaler, Azmacort™; methotrexate (MTX); interleukin antagonists such as IL-4, IL-5, IL-12 antibodies, receptor proteins or antagonists, and antagonist fusion proteins, IgE antibodies and antagonists, CD4 antagonists, antileukotrienes, platlet activating factor, thromoboxane antagonists, tryptase inhibitors, NK2 receptor antagonists, ipratropium, thephyllene, disodium chromoglycate (DSCG), functional or structural analogs thereof, and derivatives or variants thereof, and the like.

In view of the above, the IL-13R antagonist-containing powder compositions are surprisingly stable (i.e., exhibit minimal chemical and physical degradation upon preparation and storage, even under extreme conditions of temperature and humidity). The IL-13 antagonist powders of the invention (i) are readily dispersed by aerosol delivery devices (i.e., demonstrate good aerosol performance), (ii) exhibit surprisingly good physical and chemical stability during powder manufacture and processing, and upon storage, and (iii) are reproducibly prepared.

Thus, the present invention includes the unexpected discovery of chemically and physically stable spray-dried powder formulations of IL-13 antagonists such as IL-13R. IL-13R, like most other large peptides, comprises a group of proteins that bind IL-13 and are known to be particularly unstable when exposed to the shear stress, liquid-wall interactions, high temperature conditions and the like of spray drying. Surprisingly, the spray-dried powders of the invention (comprised of a plurality of spray-dried particles) exhibit bioactivity following spray drying, ostensibly indicating that higher order molecular aggregate levels and particulate aggregate levels both remain acceptably low.

The following examples are illustrative of the present invention, and are not to be construed as limiting the scope of the invention. Variations and equivalents of these examples will be apparent to those of ordinary skill in the art in light of the present disclosure, the drawings and the claims herein.

All articles, books, patents, journal articles and other publications referenced herein are hereby incorporated by reference in their entirety.

### EXAMPLES

The following Examples include the following abbreviations:

Term Definition
ACI Andersen cascade impaction
AI Active ingredient
BHR Bronchial Hyperresponsiveness
BP Blister package
BW Body Weight
% ED Percent emitted dose
ET Endotracheal
F Female
FPM Fine particle mass (in mg) of sIL-13Rα2-IgG powder from actuation of one BP with a fill weight of 5 mg, calculated by summing the total weight of the powder collected on the Andersen stages, (including the filter), with cut-off sizes <3.3 μm.
FPF_{%<3.3 μm} Fine particle fraction (proportion of particles with an aerodynamic diameter <3.3 μm)
IH Inhalation
MMAD Mass median aerodynamic diameter
MWM Molecular weight marker
PC400 Provocation Concentration
PDADS Pneumatically Driven Aerosol Delivery System
PDS Pulmonary Delivery System
PSD Particle size distribution
RH Relative humidity
R_{L} Lung Resistance
RSD Relative standard deviation
RT Retention time
SDS-PAGE Sodium dodecyl sulfate-polyacrylamide gel electrophoresis
SEC-HPLC Size-exclusion high performance liquid chromatography
SEM Scanning electron microscopy
TGA Thermogravimetric analysis

Before describing specification formulations, methods and analytical approaches will be explained.

IL-13Rα2-IgG Formulations: Spray dried IL-13Rα2-IgG particles were prepared using standard spray-drying techniques. Briefly, for each formulation, IL-13Rα2-IgG was combined with deionized water along with the stated amounts of the excipient(s) for each formulation as provided in Table 1. The total solids concentration for each formulation is also provided in Table 1. A 1% solids value indicates 10 mg/mL of solids. Typically, about 200-300 mL of liquid feed solution was prepared for each formulation. Sodium citrate and sodium hydroxide were added as necessary to provide a pH of 6.5.

Examples 1, 2, and 3 included residual amounts (e.g., about 0.1-0.2%) of Tween 80. Diafiltration (see Example 16) reduced the amount of residual Tween 80 to less than about 0.05%.

The liquid feed solution was then spray-dried using a Buchi spray dryer under the following conditions outlet temperature=60-80° C.; and flow rate=about 5 mL/minute. The powders were collected and some of the powders were characterized.

Moisture Content. The moisture content of the powders was measured by thermogravimetric analysis.

MMADs. The aerosol particle size distribution (MMAD) was determined using a cascade impactor (Graseby Andersen, Smyrna, Ga.) at a flow rate of 28 L/min, ignoring powder loss of the inlet manifold.

Emitted Dose (ED). Emitted doses were determined as described in the “Definitions” section using a dry powder inhaler as described in U.S. Pat. No. 5,740,794 and a Gelman glass filter, 47 mm diameter.

Scanning Electron Microscopy (SEM). Particle morphology was determined using an XL 30 ESEM manufactured by Philips Electron Optics (Eindhoven, The Netherlands).

### Examples 1-16

### Formulation Characterization

Table 1 lists formulations that were prepared and subsequently spray dried, with the balance of the composition being sIL-13Rα2-IgG.

TABLE 1
IL-13Rα2-IgG Formulations

| | wt/wt % | wt/wt % | wt/wt % | wt/wt % | | |
|---|---|---|---|---|---|---|
| Example | solids | sucrose | Mannitol | trileucine | Citrate | |
| 1 | 1 | 0 | 0 | 0 | 2.5 | mM |
| 2 | 1 | 30 | 0 | 0 | 0 | |
| 3 | 1 | 0 | 0 | 30 | 2.5 | mM |
| 4 | 1 | 0 | 0 | 30 | 2.5 | mM |
| 5 | 1 | 10 | 0 | 20 | 2.5 | mM |
| 6 | 1 | 15 | 0 | 20 | 5 | mM |
| 7 | 1 | 20 | 10 | 20 | 2.5 | mM |
| 8 | 1 | 30 | 0 | 20 | 2.5 | mM |
| 9 | 0.5 | 30 | 0 | 20 | 2.5 | mM |
| 10 | 1 | 0 | 0 | 15 | 2.5 | mM |
| 11 | 0.5 | 10 | 0 | 20 | 2.5 | mM |
| 12 | 0.5 | 30 | 0 | 20 | 2.5 | mM |
| 13 | 1 | 10 | 0 | 20 | 2.5 | mM |
| 14 | 0.5 | 30 | 0 | 20 | 2.5 | mM |

Characterization of Certain Spray-dried Formulations is provided in Table 2. In Table 2, Example 15 is stock solution and Example 16 is diafiltered.

TABLE 2
Characterization of IL-13Rα2-IgG Powder Formulations

| | | | | | | | SEC-% HMW | | | |
|---|---|---|---|---|---|---|---|---|---|---|
| | | | | | | | 8.2 minutes | | | |
| | | | | | | | Pre/Post Spray | | | |
| Ex. | MMADμm | FPF_{<3.3 μm} | FPF_{<4.7 μm} | FPM_{<3.3 μm} | FPM_{<4.7 μm} | ED | Drying | Active % | Dose(mg) | TGA |
| 1 | — | — | — | — | — | 14 | 2.62/3.71 | — | — | 8.5 |
| 2 | — | — | — | — | — | 8 | 2.63/3.64 | — | — | 7.3 |
| 3 | 3.2 | 0.52 | — | — | — | 15 | 2.63/3.38 | — | — | 6.1 |
| 5 | 3.5 | 0.47 | 0.80 | 1.7 | 2.8 | 81 | 6.8/5.2 | 55 | 1.2 | — |
| 9 | 2.9 | 0.60 | 0.91 | 2.3 | 3.4 | 77 | 5.9/5.4 | 37 | 0.8 | — |
| 15 | — | — | — | — | — | — | 1.59/— | — | — | — |
| 16 | — | — | — | — | — | — | 1.89/— | — | — | — |

As can been seen from Table 2, the IL-13Rα2-IgG powder formulations exhibit MMAD, FPF_{<3.3 μm}, FPF_{<4.7 μm}, FPM_{<3.3 μm}, and FPM_{<4.7 μm}, values suited for pulmonary delivery. Examples 5 and 9 also demonstrated good ED values. The determination of the formation of higher order molecular aggregates following spray drying was accomplished using size-exclusion chromatography. The values represent the measured higher order aggregate content prespray drying and again 8.2 minutes postspray drying. The results indicate good formulation stability. As IL-13Rα2-IgG was believed to associated with about 14% (by weight) carbohydrates associated with the protein, the active % (amount of active protein less any carbohydrate) in the formulation was calculated by subtracting 14% of the measured amount of IL-13Rα2-IgG in the formulation. In Examples 5 and 9, the active % amounted to 55 and 37, respectively. Nominal doses for each of these Examples were determined to be 1.2 mg and 0.8 mg, respectively. As pointed out above, these doses can be adjusted dependent on the particular needs of the given situation. Finally, the moisture content for Examples 1, 2, and 3 were all less than 10%.

### Examples 17 and 18

### SEMs of IL-13Rα2-IgG Powder Formulations

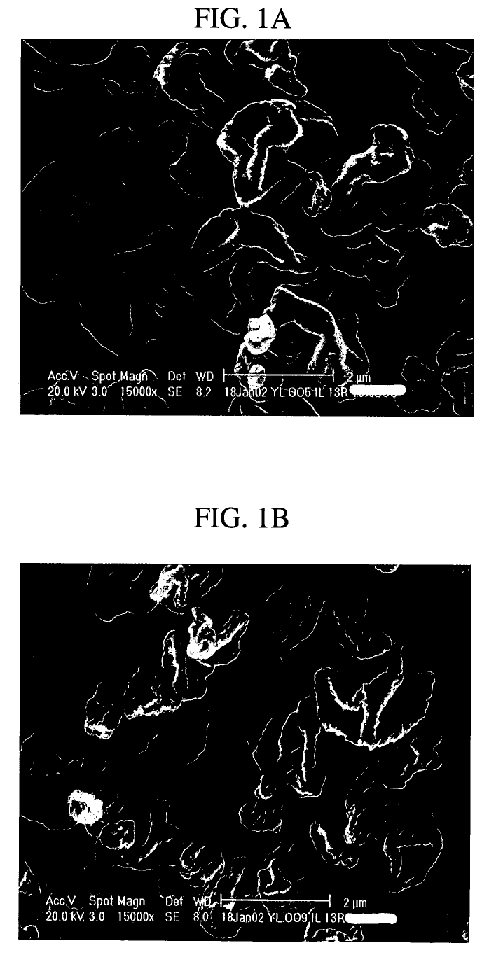

Particle morphology was determined for Examples 5 and 9. FIG. 1A corresponds to the SEM of the particles of formulation A of Example 5, while FIG. 1B corresponds to the SEM of the particles of formulation B of Example 9. In both cases, the SEMs show wrinkled, “raisin-like” shaped particles, which provide excellent aerosol properties. It is believed that the excipient trileucine plays a significant factor in providing this desired particle morphology.

### Examples 19-27

### Formulation Feasibility Assessment

### 1. SUMMARY

The objectives of these Examples were to (1) determine whether sIL-13Rα2-IgG could be formulated as a dry powder suitable for aerosol delivery using a pulmonary delivery system (PDS); and (2) identify a formulation for use in an inhalation efficacy study in a sheep model. A desirable powder was defined as one that had the following characteristics:
Percent emitted dose (% ED)>60%
Mass median aerodynamic diameter (MMAD)<3.5 μm
Fine particle fraction (FPF_{<3.3 μm})>45%
After stability storage at accelerated conditions (40° C./75% RH packaged) for 1 month: <2.5% increase in soluble aggregation and <2.5% increase in covalent aggregation with respect to the starting active pharmaceutical ingredient (API).

Nine formulations were screened, and two were chosen for full characterization (formulations A and B). Formulation A contained 55 wt % sIL-13Rα2-IgG (and corresponded to Example 5, above), and formulation B contained 37 wt % sIL-13Rα2-IgG (and corresponded to Example 9, above). The aerosol performance and physical and chemical properties of these two powders were assessed immediately after spray drying and after 1 month of stability storage. Based on the initial aerosol data, the amount of sIL-13Rα2-IgG that would potentially be delivered to the lung from one 5 mg filled blister pack (BP) of the sIL-13Rα2-IgG formulation was calculated.

Formulations A and B were manufactured for a more thorough characterization and stability evaluation. After spray drying, both formulations met the acceptance criteria. Formulation A yielded a % ED of 67%, MMAD of 2.8 μm, and FPF_{<3.3 μm}, of 64%; and formulation B yielded a % ED of 61%, MMAD of 2.4 μm, and FPF_{<3.3 μm} of 77%. Comparisons of the spray-dried powders with unsprayed protein showed that neither formulation A nor formulation B exhibited any chemical degradation after spray drying. Formulation A showed a 2.4% increase in high-molecular-weight aggregate content (with respect to the API) when exposed to 40° C., but remained within the acceptance criteria. Neither formulation exhibited any loss of aerosol performance over the time course of the stability study.

### 2. OBJECTIVE AND SCOPE

2.1 Objectives

The objectives of this project were to (1) formulate sIL-13Rα2-IgG as a dry powder for aerosol delivery; and (2) to identify a lead formulation to support an inhalation efficacy study in a sheep model.

2.2 Scope

Formulations of sIL-13Rα2-IgG were prepared and filled at 5 mg into blister packages (BPs) for evaluation using a pulmonary delivery system (PDS), as disclosed in U.S. Pat. No. 6,257,233, which is incorporated by reference herein in its entirety. The aerosol performance, solid-state properties, and chemical stability of the sIL-13Rα2-IgG formulations were evaluated after spray drying (initial time point) and after 1 month of storage at several conditions. For the stability studies, powders were filled into BPs, which were then sealed in foiled pouches with desiccant.

The target aerosol characteristics and chemical stability of the sIL-13Rα2-IgG dry powders are listed in Table 3.

TABLE 3
Target characteristics of sIL-13Rα2-IgG powders

| Variable | Designation | Value |
|---|---|---|
| Emitted dose (%) | ED | >60% |
| Mass median | MMAD | <3.5 μm |
| aerodynamic diameter | | |
| Fine particle fraction | FPF _{<3.3 μm} | >45% |
| (percent of total | | |
| particles with an | | |
| aerodynamic diameter | | |
| <3.3 μm) | | |
| Chemical stability | Aggregation | <2.5% Increase in noncovalent |
| | | aggregation and <2.5% increase |
| | | in covalent aggregation |
| | | after storage at 40° C./75% |
| | | RH for 1 month with respect |
| | | to the API starting material |

### 3. MATERIALS AND METHODS

3.1 Active Pharmaceutical Ingredient (API)

The approximate molecular weight of sIL-13Rα2α2-IgG is 142 kDa, and it is expressed as glycosylated protein. Carbohydrates constitute fourteen percent of the total mass of the sIL-13Rα2-IgG. The extinction coefficient used to determine the protein concentration was 2.18 mL mg⁻¹ cm⁻¹ at 280 nm, and was not adjusted for the effects of glycosylation.

Some earlier preliminary work with sIL-13Rα2-IgG containing Tween 80, showed that the Tween 80 had a deleterious effect on the aerosol performance of powder formulations. Thus, the lots of API of these Examples were free of Tween 80.

3.2 Formulation Preparation and Selection

Nine sIL-13Rα2-IgG formulations were prepared by diafiltering sIL-13Rα2-IgG (free of Tween 80) into a 2.5 mM citrate buffer, pH 6.5; adding excipients to enhance the aerosol performance and chemical stability of the resulting powders; and spray drying the resulting solutions using a laboratory-scale Büchi system. Initial screening of these nine powders based on aerosol performance and chemical stability for one month (powders filled in BPs and packaged in a foil pouch with desiccant) led to the selection of two formulations (formulations A and B) for development and further characterization.

The compositions (weight-by-weight; w/w) of the two lead formulations A and B are shown in Table 4. For the purpose of calculating solids content in the formulations, the active pharmaceutical ingredient (API) content shown in Table 4 refers to the proportion of the powder represented by the aglycone sIL-13Rα2-IgG; the glycan percentage is calculated as 14% of the total mass of sIL-13Rα2-IgG.

TABLE 4
Weight percentages of components in chosen test formulations

| | | Glycan from | | | |
|---|---|---|---|---|---|
| Formulation | API | sIL-13Rα2-IgG | Sucrose | Trileucine | Citrate |
| Designation | (%) | (%) | (%) | (%) | Buffer (%) |
| Formulation A | 55 | 9 | 10 | 20 | 6 |
| Formulation B | 37 | 6 | 30 | 20 | 6 |

3.3. Formulation Evaluation
3.3.1 Stability Testing

To test the stability of the sIL-13Rα2-IgG in the test formulations over 1 month, the powders were filled into blister packs (BPs) at 5 mg total fill weight. The BPs were then sealed in foil pouches with desiccant and stored in incubation chambers under two sets of conditions. Additional powder samples (referred to as “unpackaged” samples) were placed in uncapped glass vials and were stored unprotected under conditions of controlled temperature and humidity. The aerosol tests of the packaged powders were performed using the stored BPs, and the chemical tests were performed on reconstituted solutions of the packaged powder (BPs) and unpackaged powder (bulk). These analyses were conducted at the initial time and after 1 month of storage under the conditions indicated in Table 5.

TABLE 5
Stability protocol

| | | | BPs in Pouch with Desiccant | | Bulk Powder |
|---|---|---|---|---|---|
| | | | 1 month @ | 1 month @ | 1 month @ |
| Parameter | Assay | Initial | 25° C./60% RH | 40° C./75% RH | 25° C./60% RH |
| Aerosol performance | ED | X | X | X | — |
| | MMAD | X | X | X | — |
| Chemical | SEC | X | X | X | X |
| | SDS-PAGE | X | X | X | X |
| | UV | X | X | X | X |
| Residual solvent | TGA | X | X | X | — |
| Gross morphology | SEM | X | X | X | X |

The specific methods used to characterize the aerosol performance and assess the stability of sIL-13Rα2-IgG are listed in Table 6.

TABLE 6
Methods used to characterize sIL-13Rα2-IgG formulations

| | |
|---|---|
| Parameter | Method |

Aerosol Performance Analyses

| | |
|---|---|
| Emitted dose (ED) | Gravimetric analysis, flow |
| | rate = 30.0 L/min (n = 10) |
| Particle size distribution | Gravimetric-based Andersen |
| (PSD): Mass median aerodynamic | cascade impaction (ACI) |
| diameter (MMAD), fine particle | (stage cut-off sizes: 9, 5.8, |
| fraction (FPF_{<3.3 μm}), fine | 4.7, 3.3, 2.1, 1.1, 0.7, and |
| particle dose (FPD_{<3.3 μm}) | 0.4 μm, and filter) at a |
| | flow rate of 28.3 L/min (n = 3) |

Solid-State Analyses

| | |
|---|---|
| Gross morphology | Scanning electron microscopy |
| | (SEM), Au/Pd sputter coating |

Chemical Analyses

| | |
|---|---|
| Moisture content | Thermogravimetric analysis (TGA) |
| Degradation and aggregation | Size-exclusion chromatography |
| | (SEC): (total soluble) |
| | aggregation |
| | SDS-PAGE: covalent aggregation |
| | and degradation |

3.3.2 Aerosol Powder Performance

The aerosol performance of each of the powders was determined using a PDS inhaler, as disclosed in U.S. Pat. No. 6,257,233, which is incorporated by reference herein in its entirety. Aerosol performance was evaluated by gravimetrically determining the percent emitted dose (% ED; the percentage of BP fill weight emitted from the inhaler after the actuation of one BP), and by determining the particle size distribution (PSD) of the formulations filled into the BPs using an Andersen cascade impactor (ACI). PSD parameters included mass median aerodynamic diameter (MMAD), fine particle fraction (FPF_{<3.3 μm}; percentage of delivered particles with aerodynamic diameters less than 3.3 μm;), and fine particle dose (FPD_{<3.3 μm}; the mass of aglycone sIL-13Rα2-IgG API delivered in particles <3.3 μm).

From the various historical human clinical studies using both gamma scintigraphy and pharmacokinetics, the amount of sIL-13Rα2-IgG that would be delivered to the lung in humans was estimated as follows for the PDS:
Drug_{lung}=φ_{L}×φ_{ED}×BP_{fill}×Wt_{AI} Equation 1
where φ_{L} is the fraction deposited in the human lung, φ_{ED} is the emitted dose; BP_{fill} is the fill weight of the BP, and Wt_{AI} is the weight percent active ingredient in the formulation.
3.3.3 Physical and Chemical Assessment

The gross morphology of the particles was assessed by scanning electron microscopy (SEM), and the chemical stability of the powders was evaluated by size exclusion chromatography (SEC) for total soluble aggregation and sodium dodecyl sulfate-polyacrylamide gel electrophoresis (SDS-PAGE) for covalent aggregation. Moisture content of the powders was determined by thermogravimetric analysis (TGA), by heating samples to 110° C. at 10° C./min and holding the temperature at 110° C. for 20 minutes.

### 4. RESULTS

After spray drying and packaging (filling into BPs and sealing into foil pouches with desiccant), and after 1 month of stability storage under the test conditions, both sIL-13Rα2-IgG formulations met the target powder characteristics listed in Table 3.

4.1 Aerosol Performance

The aerosol performance results from the testing of the sIL-13Rα2-IgG formulations are listed in Table 7. For formulation A, the initial and one-month % ED values ranged from 67 to 70%; the initial and one-month MMAD values ranged from 2.6 to 2.8 μm; the initial and one-month FPF_{<3.3 μm} values ranged from 64 to 71%; and the initial and one-month FPD_{<3.3 μm} values ranged from 1.4 to 1.7 mg. For formulation B, the initial and one-month % ED values ranged from 61 to 63%; the initial and one-month MMAD values ranged from 2.3 to 2.4 μm; the initial and one-month FPF_{<3.3 μm} values ranged from 77 to 78%; and the initial and one-month FPD_{<3.3 μm} values were 1.1 mg.

No changes in aerosol performance were observed in either formulation A or B, under any of the stability conditions tested.

TABLE 7
Aerosol performance of sIL-13Rα2-IgG powders immediately after spray
drying (initial) and after 1 month of storage under indicated conditions

| | | | | PSD Data | | |
|---|---|---|---|---|---|---|
| | | % ED @ 30 L/min | | MMAD | FPF_{<3.3 μm} | FPD_{<3.3 μm} |
| | Stability | Mean | | (μm) | (%) | (mg) |
| Formulation | Conditions | (n = 10) | RSD^{a} | (n = 3) | (n = 3) | (n = 3) |
| Target | n/a | >60 | — | <3.5 | >45 | — |
| A | Initial | 67 | 5 | 2.8 | 64 | 1.7 |
| | 25° C./60% RH/ | 70 | 5 | 2.7 | 66 | 1.7 |
| | desiccated | | | | | |
| | 40°/75% RH/ | 70 | 6 | 2.6 | 71 | 1.4 |
| | desiccated | | | | | |
| B | Initial | 61 | 8 | 2.4 | 77 | 1.1 |
| | 25° C./60% RH/ | 63 | 5 | 2.3 | 78 | 1.1 |
| | desiccated | | | | | |
| | 40° C./75% RH/ | 62 | 6 | 2.3 | 78 | 1.1 |
| | desiccated | | | | | |

^{a}RSD = relative standard deviation

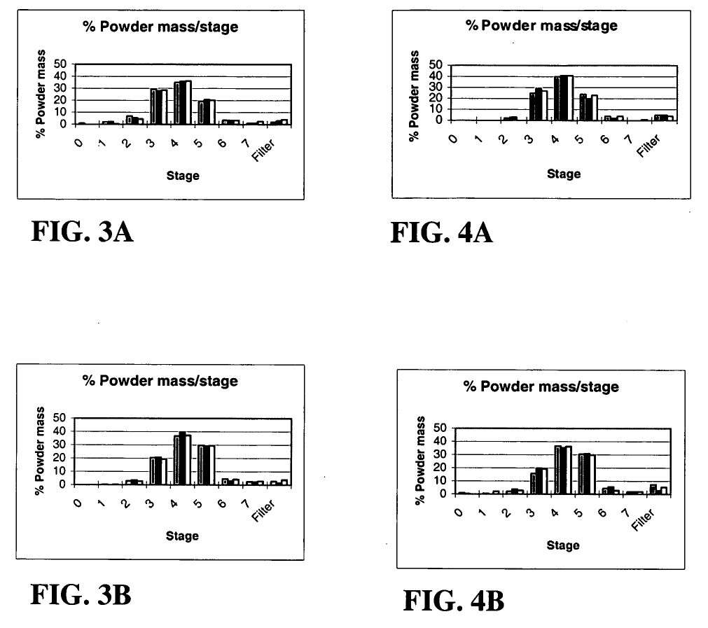

The particle size distribution profiles of the powders are shown in FIGS. 3A, 3B, 4A, and 4B, wherein the particle size distribution profiles were determined using the particle size cutoffs shown in Table 8. FIG. 3A shows the initial particle distribution profile for formulation A, and FIG. 4A shows the particle distribution profile for formulation A after storage in BPs stored in foil pouches for 1 month at 40° C. and 75% relative humidity with desiccant for 1 month. FIG. 3B shows the initial particle distribution profile for formulation B, and FIG. 4B shows the particle distribution profile for formulation B after storage in BPs stored in foil pouches for 1 month at 40° C. and 75% relative humidity with desiccant for 1 month.

TABLE 8
Particle Size Cutoffs

| | Particle Size Cutoff |
|---|---|
| Stage | (μm) |
| 0 | 9.0 |
| 1 | 5.8 |
| 2 | 4.7 |
| 3 | 3.3 |
| 4 | 2.1 |
| 5 | 1.1 |
| 6 | 0.7 |
| 7 | 0.4 |
| Filter | <0.4 |

Table 9 shows the predicted lung deposition of the test sIL-13Rα2-IgG formulations calculated according to Equation 1.

TABLE 9
Predicted lung deposition of sIL-13Rα2-IgG
from actuation of one 5 mg BP

| | Predicted Lung |
|---|---|
| Formulation | Deposition (mg)^{a} |
| A | 1.1 |
| B | 0.7 |

^{a}Based on actuation of one BP with a fill weight of 5 mg (Equation 1).

4.2 Morphology

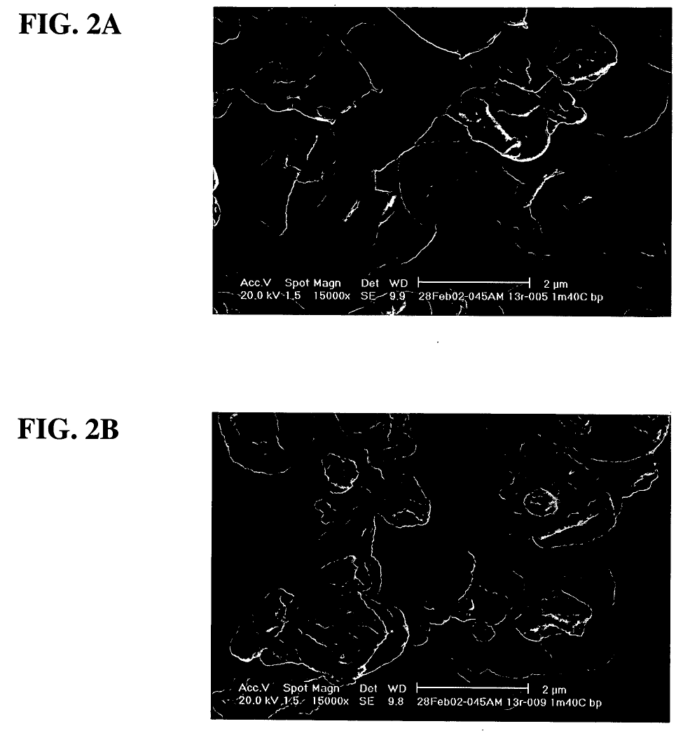

FIGS. 2A and 2B are SEM images of formulations A and B, respectively, after 1 month of storage at 40° C./75% RH in BPs sealed in foil pouches with desiccant, there were no visible changes in gross morphology of any of the test powders.

4.3 Chemical Stability

4.3.1 Moisture Content

Table 10 shows the moisture content of each formulation after spray drying and after 1 month of storage in BPs sealed in foil pouches with desiccant. A loss of moisture from the powders was observed during storage, presumably due to the low humidity environment created in filling and storage.

TABLE 10
Moisture content (%) of sIL-13Rα2-IgG formulations

BPs in pouch with desiccant

| | | 1 month @ | 1 month @ |
|---|---|---|---|
| Formulation | Initial | 25° C./60% RH | 40° C./75% RH |
| A | 5.0 | 3.7 | 1.4 |
| B | 3.0 | 1.8 | 1.7 |

4.3.2 Aggregation Measured by SEC

SEC analyses were performed on bulk powder exposed to controlled temperature and humidity conditions, as well as on powders that had been filled into BPs and sealed with desiccant in foil pouches.

The size-exclusion chromatograms indicated no increase in soluble aggregate formation in the packaged samples at 25° C./60% RH or in formulation B at 40° C./75% RH. However, the packaged samples of formulation A stored at 40° C./75% RH, and the unpackaged samples of both formulations exposed directly to elevated temperatures and RH conditions showed evidence of soluble aggregate formation, with soluble aggregates accounting for up to 2.4% with respect to the reference API. Nevertheless, both formulations A and B stored at 40° C./75% RH packaged with desiccant for 1 month met the target criteria of <2.5% increase in soluble aggregate content relative to the API.

4.3.3 Aggregation by SDS-PAGE

SDS-PAGE showed no evidence of sIL-13Rα2-IgG degradation relative to the API, or of covalent aggregate formation, in any of the packaged samples. SDS-PAGE also showed no evidence of covalent aggregate formation in any of the packaged or unpackaged stability storage samples.

### 5. CONCLUSIONS AND RECOMMENDATIONS

The aerosol performance of the two sIL-13Rα2-IgG formulations met the predetermined acceptance criteria.

The aerosol and chemical properties of the sIL-13Rα2-IgG formulations were stable to the spray-drying process and to 1 month of storage in both foil-wrapped BPs with desiccant at temperatures up to 40° C. and bulk powder exposed to RH values up to 60% at 25° C.

The dose in the sheep model required approximately 0.2 mg to be delivered to the lung. Both formulations met the target characteristics, however formulation A has the higher predicted deposition, which suggested proceeding with formulation A for an inhalation efficacy study in sheep. Formulation B was shown to be more chemically stable than Formulation A.

### Comparative Examples 1-3

### Determination of the Tolerability of Dry Powder Vehicle Alone Over Dose Range Representative of Dry Powder Required to Deliver Low (0.14 mg/kg) to High (0.5 mg/kg) Daily for Two Days Dose of Active Substance

### 1. ABSTRACT

Three asthmatic sheep were given increasing doses of inhaled vehicle dry powders to determine the maximum tolerated dose and to determine the tolerability of the dose to be used in an efficacy study with sIL-13Rα2-IgG. Two different vehicle powders were used in this study, vehicle-1 and vehicle-2. Vehicle-1 contained the excipient citrate and vehicle-2 did not contain citrate. The maximum tolerated dose was defined as the amount of powder that caused a 100% increase in lung resistance relative to baseline. Increasing lung dose of vehicle dry powder (vehicle-1 or vehicle-2) to higher doses caused an increase in lung resistance in the asthmatic sheep. In this limited study, there was a difference in the tolerability of vehicle between the two different formulations. The dose that caused a 100% increase in lung resistance in this model was approximately 19 mg of vehicle-1 powder (0.38 or 0.54 mg/kg) and approximately 37 mg of vehicle-2 powder (0.91 mg/kg). Inhalation of dry powder vehicle-1 did not affect the non-specific bronchial hyperresponsiveness (BHR) to carbachol. The lung response was low (<50%) and transient (returned to baseline in 5 min) at a dose of 2 blister packs (estimated total powder dose of approximately 10 mg).

### 2. OBJECTIVE

The objective of this study was to determine the maximum tolerated dose of dry powder vehicle in a sheep model of asthma. This study was performed in preparation for testing the efficacy of a dry powder formulation of sIL-13Rα2-IgG in the sheep model of asthma. Bronchoconstriction is the measured parameter in the sheep asthma model.

### 3. STUDY DESIGN

Three sheep were used in this study. Two sheep were given escalating doses (1 to 8 blister packs, BPs) of vehicle-1. Vehicle-1 contains citrate. Lung resistance (R_{L}) was measured before and after dose delivery. After 15 min, or when resistance returned to baseline, the next dose was administered. Dose escalation was stopped when lung resistance increased by 100% over baseline. 24 hours after the dose-escalation, the non-specific bronchial hyperresponsiveness (BHR) to carbachol was measured and compared to baseline results.

The dose escalation procedure was also performed with a third sheep. This time a different vehicle (vehicle-2) was used. Vehicle-2 did not contain the excipient citrate. The BHR response was not measured in this sheep.

### 4. MATERIALS

4.1 Test System and Animal Husbandry

Adult female sheep (n=3; body weight, BW=35-50 kg) were used for these studies. These sheep had been tested previously for BHR using carbachol. Two female sheep were used for Group 1 and 1 female sheep was used for Group 2 (total of 3 female sheep).

4.1.1 Species

Sheep

4.1.2 Number/Gender Per Group

2 F/Group 1 and 1 F/Group 2

4.1.3 Housing

The sheep were housed on a 12-hour light/12-hour dark cycle in pens at the laboratory animal facility. The room temperature and relative humidity were not monitored.

4.1.4 Identification

Each sheep was uniquely identified by an ear tag, as well as on a shaved area of the animal's flank with an indelible marker.

4.1.5 Body Weights

Each sheep was weighed prior to the study. The animals weighed 35, 41, and 50 kg.

4.1.6 Disposition of Animals

These experiments were not designed to measure death as an endpoint. No animal exhibited signs of systemic toxicity or distress. Animals recovered from the study and were placed back into the animal pool to be used in future studies.

5.2 Control Articles

5.2.1 Control Article-Vehicle-1

Chemical/Common Name: Vehicle-1
Description: See Table 11
Storage: Blister pack sealed in foil pouches with desiccant. Ambient storage conditions.
   5.2.2 Control Article-Vehicle-2
Chemical/Common Name: Vehicle-2
Description: See Table 11
Storage: Blister pack sealed in foil pouches with desiccant. Ambient storage conditions.

### 6. METHODS

6.1 Formulation

The formulations were prepared using the procedure of Examples 1-16, except that the compositions (weight-by-weight; w/w) of the two formulations are shown in Table 11.

TABLE 11
Composition of vehicle control formulations

| | Nominal BP | % Excipient (w/w) | | | |
|---|---|---|---|---|---|
| Formulation | Fill Weight | | | | Citrate |
| Designation | (mg) | Sucrose | Trileucine | Leucine | Buffer |
| Vehicle-1 | 5.00 | 10 | 20 | 64 | 6 |
| Vehicle-1 | 7.50 | 10 | 20 | 64 | 6 |
| Vehicle-2 | 7.50 | 0 | 26 | 74 | 0 |

6.2 Dry Powder Delivery Procedure

The study was performed over 2 days in Florida. On the first day, a pneumatically driven aerosol delivery system (PDADS) was set up. The PDADS involved a PDS, as disclosed in U.S. Pat. No. 6,257,233, which is incorporated by reference herein in its entirety, connected to a Harvard Apparatus large animal respirator. In vitro efficiency of the PDADS was determined by the mass of powder delivered through an endotracheal (ET) tube. The results were compared to those obtained on the system in California prior to shipment to the study site. In vitro system efficiency measurements were repeated daily. The ventilatory parameters used were: 500 mL tidal volume, 5 breaths per minute, and 50:50 inspiratory: expiratory cycle. The operational steps that were used to deliver dry powder aerosol to the sheep lung were:
1. When the ventilator was at the end of inhalation, the blister was placed in the PDS device and aerosolized into the dispersion chamber.
2. The pneumatic piston was then switched into alignment with the dispersion chamber.
3. When the ventilator began the inspiratory cycle (as determined by activation of the dosimeter that was synchronized to fire when the ventilator began inspiration) the pneumatic piston was activated to deliver the powder to the sheep.
4. Another full inhalation cycle was allowed before the system was disconnected from the ET tube and the piston retracted from the cylinder.
5. Steps 1-4 were repeated if more than one blister was delivered

Vehicle dry powder aerosols were delivered to the conscious restrained sheep. The sheep were intubated and the ET tube was attached to the PDADS and the Harvard ventilator. After about 2 minutes when the sheep became accustomed to the system, the dry powder delivery was initiated.

6.3 Physiologic Response

The physiologic response to the inhaled powder was measured as the percent change in lung resistance (R_{L}) relative to baseline. 24 hours following vehicle-1 delivery, BHR was determined using carbachol. The dose of carbachol required to achieve a 400% change in R_{L} (PC400) 24 hrs following vehicle was compared to historic PC400 values to determine if the vehicle had any effect on airway responsiveness. See Abraham et al., A4-Integrins mediate antigen-induced late bronchial responses and prolonged airway hyperresponsiveness in sheep. J. Clin. Invest. 93:776-787, 1994, which is incorporated by reference herein in its entirety.

6.4 Dose Escalation Procedure

Baseline R_{L} was measured on each sheep and then the first dose of vehicle (1 or 2) was administered (dose 1, see Table 12). R_{L} was again measured over the next 10 min. After 15 min, or when R_{L} returned to baseline, dosing was repeated with a second, higher dose of vehicle (1 or 2). This dose-response sequence was repeated until R_{L} increased to 100% over baseline.

TABLE 12
Target estimated dose based on in vitro evaluation

| | Number | | Target Estimated | Target Estimated |
|---|---|---|---|---|
| | of | Target BP Fill | Lung Powder | Lung Powder Dose |
| Dose | BPs | Mass (mg) | Dose^{a} (mg) | (mg/kg) |
| 1 | 1 | 5.00 or 7.50 | 2.50 or 3.75 | 0.08 or 0.13 |
| 2 | 1 | 7.50 | 3.75 | 0.13 |
| 3 | 2 | 7.50 | 7.50 | 0.25 |
| 4 | 4 | 7.50 | 15.00 | 0.50 |
| 5 | 8 | 7.50 | 30.00 | 1.00 |

Notes:
BP(s) = Blister pack(s);
Estimated delivery efficiency = 50%;
BW = 30 kg
^{a}Estimated Dose = (Target BP Fill mass, mg) × (# BPs) × Delivery Efficiency

6.5 Dose Estimation

The PDADS system was characterized to determine the in vitro system efficiency for study planning and to estimate the target dose to be delivered to the sheep (see Table 12). The efficiency measurements were not performed using the ventilator but by using a house air source to add a chase air bolus to deliver the powder to the filter.

The PDADS system efficiency was again estimated by using the Harvard ventilator, and these measurements were used to estimate the dose delivered at the study site. For the in vitro estimates, a filter (glass fiber) was placed at the end of the ET tube to collect the powder delivered. Efficiency is the mass deposited on the filter divided by the BP fill mass times 100. The powder dose deposited on the filter was determined gravimetrically. Five separate efficiency measurements (1 BP per measurement) were made each day. The average efficiency of the system measured 5 times on at least 2 experimental days was used to estimate delivered dose to the sheep.

6.6 Group Assignments

Table 13 summarizes the treatment regimens of the two groups.

TABLE 13
Summary of treatment regimens for sheep in groups 1 and 2

| | | | | No of |
|---|---|---|---|---|
| Group | Comparative | Control and Test | Route of | Dosing |
| No. | Example | Article | Administration | Days |
| 1 | 1, 2 | Vehicle-1 | IH | 1 |
| 2 | 3 | Vehicle-2 | IH | 1 |

### 7. RESULTS AND DISCUSSION

7.1 Aerosol System Efficiency

The average delivery efficiency of the PDADS system was 64±6% for vehicle-1 as measured on three consecutive days of testing at the study site. Using this efficiency number, the estimated dose of vehicle delivered to each sheep is listed in Table 14, below. The estimated dose delivered was escalated from approximately 3 to 19 mg (0.07 to 0.54 mg/kg).

TABLE 14
Estimated quantity of vehicle-1 delivered per dose to the sheep

| Comparative | | | | No. BPs Per | Nominal Dose ^{a} | Estimated Lung Dose | |
|---|---|---|---|---|---|---|---|
| Example | Sheep # | BW (kg) | Dose # | Dose | (mg) | mg ^{b} | mg/kg ^{c} |
| 1 | 2016 | 50 | 1 | 1 | 5.08 | 3.25 | 0.07 |
| | | | 2 | 1 | 7.45 | 4.77 | 0.10 |
| | | | 3 | 2 | 14.86 | 9.51 | 0.19 |
| | | | 4 | 4 | 29.94 | 19.16 | 0.38 |
| | | | Cumulative | 8 | 57.33 | 36.69 | 0.73 |
| 2 | 2018 | 35 | 1 | 1 | 5.05 | 3.23 | 0.09 |
| | | | 2 | 1 | 7.46 | 4.77 | 0.14 |
| | | | 3 | 2 | 15.06 | 9.64 | 0.28 |
| | | | 4 | 4 | 29.78 | 19.06 | 0.54 |
| | | | Cumulative | 8 | 57.35 | 36.70 | 1.05 |

Assumption: IH delivery efficiency = 64%
^{a} Nominal Dose (mg) = Actual BP powder fill mass (mg) × number of BPs
^{b} Estimated Lung dose (mg) = Nominal Dose (mg) × IH delivery efficiency
^{c} Estimated Lung Dose (mg/kg) = Estimated Lung Dose (mg)/BW (kg)

The aerosol delivery efficiency of the PDADS for vehicle-2 was 62+2%. Using this efficiency number, the estimated dose of vehicle-2 delivered to each sheep is listed in Table 15, below. The estimated dose delivered for vehicle-2 was escalated from approximately 5 to 37 mg (0.12 to 0.91 mg/kg).

TABLE 15
Estimated quantity of vehicle-2 delivered per dose to the sheep

| Comparative | | | | No. BPs | Nominal Dose ^{a} | Estimated Lung Dose | |
|---|---|---|---|---|---|---|---|
| Example | Sheep # | BW(kg) | Dose # | Per Dose | (mg) | mg ^{b} | mg/kg ^{c} |
| 3 | 2048 | 41 | 1 | 1 | 7.65 | 4.74 | 0.12 |
| | | | 2 | 1 | 7.61 | 4.72 | 0.12 |
| | | | 3 | 2 | 14.95 | 9.27 | 0.23 |
| | | | 4 | 4 | 30.11 | 18.67 | 0.46 |
| | | | 5 | 8 | 59.90 | 37.14 | 0.91 |
| | | | Cumulative | 16 Total | 120.22 | 74.54 | 1.82 |

Assumption: IH delivery efficiency = 62%
^{a} Nominal Dose (mg) = Actual BP powder fill mass (mg) × number of BPs
^{b} Estimated Lung dose (mg) = Nominal Dose (mg) × IH delivery efficiency
^{c} Estimated Lung Dose (mg/kg) = Estimated Lung Dose (mg)/BW (kg)

7.2 Effect of Dose on R_{L}

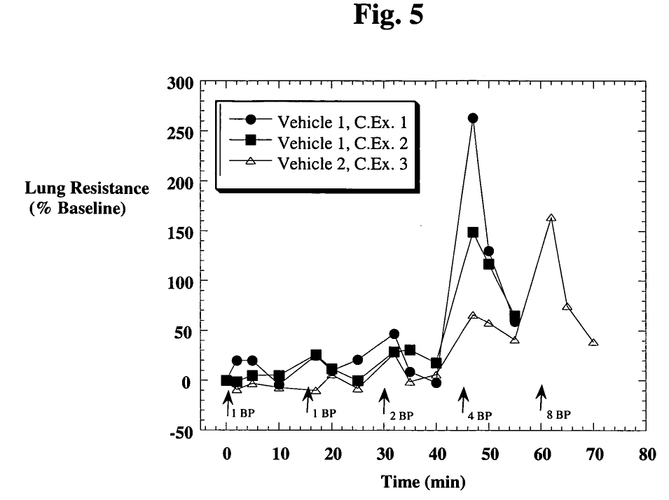

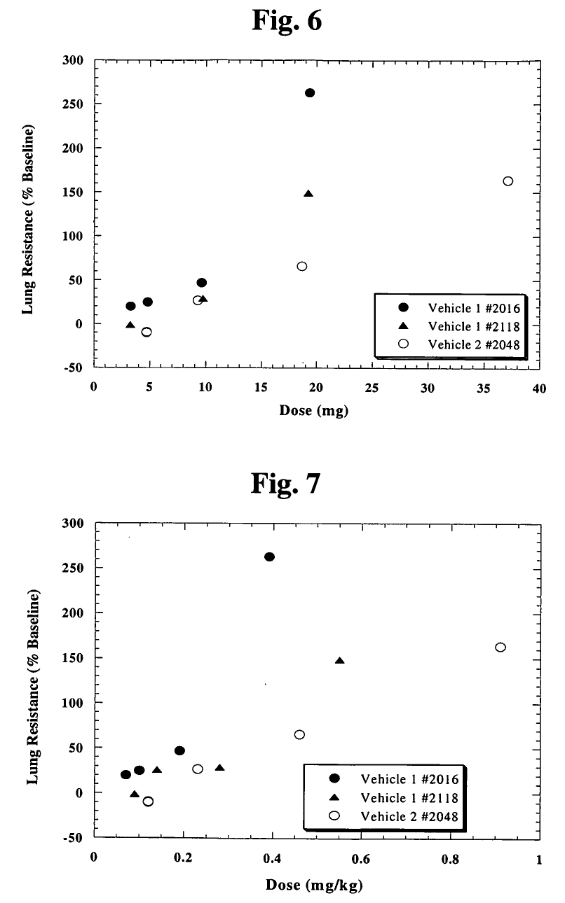

Administration by inhalation of 1 or 2 BPs (approximately 3 to 10 mg; 0.07 to 0.28 mg/kg) of vehicle-1 vehicle-2 dry powder had no effect or caused only a modest increase in R_{L} (<50% increase over baseline, FIG. 5). A higher dose of 4 BPs of vehicle-1 (approximately 19 mg; 0.38 or 0.54 mg/kg) caused a more than 100% increase in R_{L}. The response to a similar dose of vehicle-2 was less (about 50% change in R_{L}). FIGS. 6 and 7 show the relationship between dose and the immediate increase in R_{L} in the asthmatic sheep. Resistance increases with increasing dose for both vehicle-1 and vehicle-2. The dose that caused at least 100% increase in lung resistance in this model was approximately 19 mg for vehicle-1 (0.38 or 0.54 mg/kg) and approximately 37 mg for vehicle-2 (0.91 mg/kg).

BHR was measured 24 hours after the delivery of the dry powder. This measurement was performed in only the two sheep that received vehicle-1. No difference was noted compared to the historic control.

### 8. CONCLUSIONS

Increasing the inhaled dose of dry powder vehicle to high doses elicits a bronchoconstrictive response (i.e., an increase in R_{L}) in the asthmatic sheep. In this study, there was a difference in the tolerability of different formulations to elicit the response. The dose that caused a 100% increase in lung resistance in this model was approximately 19 mg for vehicle-1 (0.38 or 0.54 mg/kg) and approximately 37 mg of vehicle-2 (0.91 mg/kg). The efficacy study for sIL-13Rα2-IgG formulated with vehicle-1 required only 2 blister packs (BPs, estimated target powder lung dose of approximately 10 mg). The lung response was low (<50%) and transient (returned to baseline in 5 min) at this dose. The transient response to inhaling approximately 10 mg of dry powder should not interfere with the interpretation of the efficacy study for sIL-13Rα2-IgG.

### Examples 28 and 29, and Comparative Example 4

### Pharmacokinetics and Pharmacodynamics of sIL-13Rα2-IgG 0.2 mg/kg in Sheep after Inhalation

### 1. ABSTRACT

Sheep were treated with either inhaled vehicle (n=1) or sIL-13Rα2-IgG (active; n=2) dry powder to determine efficacy in an antigen challenge model of asthma. Two doses of dry powder were administered by inhalation 24 hrs and again at 2 hrs prior to antigen challenge. The total amount of dry powder vehicle delivered per dose was approximately 10 mg (0.27 mg/kg). The total amount of dry powder active delivered per dose was approximately 5 mg sIL-13Rα2-IgG (0.14 mg/kg). The change in lung resistance was measured over the next 8 hrs. Twenty four hrs post-antigen challenge the non-specific bronchial hyperresponsiveness (BHR) to carbachol was measured and results were compared to prior control data. Treatment with inhaled vehicle had no effect on BHR or the response to antigen challenge. Treatment with 0.14 or 0.15 mg/kg of inhaled sIL-13Rα2-IgG to the sheep at 24 and again at 2 hrs prior to antigen challenge (0.28 or 0.30 mg/kg cumulative dose) inhibited the late asthmatic response and decreased BHR.

### 2. OBJECTIVE

The objective of this study was to determine the efficacy and pharmacokinetics of dry powder sIL-13Rα2-IgG in a sheep model of asthma.

### 3. STUDY DESIGN

Three sheep were treated with an inhaled (IH) dry powder formulation of either vehicle or sIL-13Rα2-IgG at 24 hrs or 2 hrs prior to antigen challenge. Lung resistance (R_{L}) was measured before and immediately after dose delivery and again just before antigen challenge (time 0). Then, the sheep were exposed to aerosolized antigen (ascaris sum). R_{L} was measured over the next 8 hrs. 24 hours after the dose-escalation, BHR to carbachol was measured and compared to historic baseline data.

### 4. MATERIALS

4.1 Test System and Animal Husbandry

Adult female sheep (n=3; BW=35-37 kg) were used for these studies. These sheep had been tested previously for BHR using carbachol. One female sheep was used for Group 1, and 2 female sheep were used for Group 2 (total of 3 female sheep). The sheep were housed in the lab animal facility.

4.1.1 Species

Sheep

4.1.2 Number/Gender Per Group

1 F/Group 1 and 2 F/Group 2

4.1.3 Housing

The sheep were housed on a 12-hour light/12-hour dark cycle in pens at the laboratory animal facility. The room temperature and relative humidity were not monitored.

4.1.4 Identification

Each sheep was uniquely identified by an ear tag, as well as on a shaved area of the animal's flank with an indelible marker.

4.1.5 Body Weights

Each sheep was weighed prior to the study. The animals weighed between 35-37 kg.

4.1.6 Disposition of the Animals

These experiments were not designed to measure death as an endpoint. No animal exhibited signs of systemic toxicity or distress. The animals recovered from the study and were placed back into the animal pool to be used in future studies.

4.2 Control and Test Articles

4.2.1 Control Article-Vehicle

Chemical/Common Name: Vehicle 1
Description: See Table 16
Storage: Blister pack sealed in foil pouches with desiccant. Ambient storage conditions.
   4.2.2 Test Article-Active
Chemical/Common Name: sIL-13Rα2-IgG
Description: See Table 16
Storage: Blister pack sealed in foil pouches with desiccant. Ambient storage conditions.

### 5. METHODS

5.1 Formulation

Preparation of the formulations was the same as Examples 1-16, except that the compositions (% weight-by-weight; % w/w) of the two formulations are shown in Table 16. For the purpose of this report and for calculating dose and solids content in the formulations, the active pharmaceutical ingredient (API) content shown in Table 16 refers to the proportion of the powder represented by the aglycone (non-carbohydrate) part of sIL-13Rα2-IgG; the glycone percentage is calculated as 14% of the total mass of sIL-13Rα2-IgG.

TABLE 16
Composition of the formulations (% w/w)

| | | | | | | Citrate |
|---|---|---|---|---|---|---|
| Formulation | | Glycone from | | Trileucine | Leucine | Buffer |
| Designation | API (%) | sIL-13Rα2-IgG (%) | Sucrose (%) | (%) | (%) | (%) |
| sIL-13Rα2-IgG | 55 | 9 | 10 | 20 | 0 | 6 |
| Vehicle | 0 | 0 | 10 | 20 | 64 | 6 |

5.2 Dry Powder Delivery Procedure

The study was performed over 3 days. On the preceding day, the tolerability of the sheep to the dose of inhaled powder to be used in this study was tested, as described in Comparative Examples 1-3. Dry powder aerosols (vehicle or active) were delivered to the conscious, restrained sheep. Sheep were intubated and the endotracheal (ET) tube was attached to a pneumatically driven aerosol delivery system (PDADS) connected to a large animal ventilator (Harvard Apparatus). After about 2 minutes, when the sheep became accustomed to the system, the dry powder delivery was initiated.

b 5.3 Physiologic Response

The physiologic response to antigen challenge was measured as the percent change in lung resistance (R_{L}) relative to baseline. 24 hrs following antigen challenge, BHR was determined using carbachol. The dose of carbachol required to achieve a 400% change in R_{L} (PC400) 24 hrs following antigen challenge was compared to historic PC400 values to determine if the treatment had any effect on airway responsiveness. The details of this technique are disclosed in Abraham et al., A4-Integrins mediate antigen-induced late bronchial responses and prolonged airway hyperresponsiveness in sheep. J. Clin. Invest. 93:776-787, 1994, which is incorporated by reference herein in its entirety.

5.4 Dose Estimation

The PDADS system was characterized to determine the in vitro system efficiency for study planning and to estimate the target dose to be delivered to the sheep (see Table 17). The efficiency measurements were not performed using the ventilator but by using a house air source to add a chase air bolus to deliver the powder to the filter.

The PDADS system efficiency was also estimated using the Harvard ventilator and these measurements were used to estimate the dose delivered at the study site. For the in vitro estimates, a filter (glass fiber) was placed at the end of the ET tube to collect the powder delivered. The powder dose deposited on the filter was determined gravimetrically. Efficiency is the mass deposited on the filter divided by the BP fill mass times 100. Five separate efficiency measurements (1 BP per measurement) were made each day. The average efficiency of the system measured 5 times on at least 2 experimental days was used to estimate delivered dose to the sheep.

TABLE 17
Target estimated dose based on in vitro evaluation

| | | | Target Estimated Lung Powder | | Target Estimated API Lung | |
|---|---|---|---|---|---|---|
| | No. | Nominal | Dose | | Dose | |
| Group | BPs/Dose | Dose (mg) ^{a} | mg ^{b} | mg/kg ^{c} | mg ^{d} | mg/kg ^{c} |
| Vehicle | 2 | 15.00 | 7.50 | 0.25 | 0.00 | 0.00 |
| sIL-13Rα2-IgG | 2 | 15.00 | 7.50 | 0.25 | 4.13 | 0.14 |

Assumptions:
50% IH delivery efficiency;
7.50 mg target BP powder fill mass;
55% API per blister;
BW = 30 kg
^{a} Nominal Dose (mg) = Target BP powder fill mass (mg) × #BP
^{b} Estimated Vehicle Lung Dose (mg) = Nominal dose (mg) × IH delivery efficiency
^{c} Estimated Lung Dose (mg/kg) = Estimated Lung Dose (vehicle or API, mg)/BW (kg)
^{d} Estimated API Dose (mg) = Nominal dose (mg) × 0.55 (fraction of API in total powder) × IH delivery efficiency

5.5 Group Assignments

Table 18 summarizes the treatment regimens of the two groups.

TABLE 18
Summary of treatment regimens for sheep in groups 1 and 2

| | | | | No. of |
|---|---|---|---|---|
| Group | | Control and Test | Route of | Dosing |
| No. | Sheep Ids^{a} | Article | Administration | Days |
| 1 | 2116 | Vehicle | IH | 2 |
| 2 | 2119, 2121 | sIL-13Rα2-IgG | IH | 2 |

^{a}All sheep were females.

### 6. RESULTS AND DISCUSSION

6.1 Aerosol System Efficiency

The average delivery efficiency of the PDADS system was 64±6% (Mean±RSD) for the vehicle as measured on three consecutive days of testing at the study site. The average efficiency of the PDADS system for the sIL-13Rα2-IgG dry powder was 64±5% as measured on two consecutive days of testing at the study site. Using these efficiency numbers, the estimated dose of vehicle and sIL-13Rα2-IgG delivered to each sheep was calculated and is listed in Table 19 below.

TABLE 19
Estimated dose of vehicle and sIL-13Rα2-IgG delivered to the sheep

| | | | | | Estimated Powder | | Estimated API | |
|---|---|---|---|---|---|---|---|---|
| Sheep | BW | | No. BPs | Nominal | Lung Dose | | Lung Dose | |
| ID | (kg) | Day | Per Dose | Dose (mg)^{a} | (mg)^{b} | (mg/kg)^{c} | (mg)^{d} | (mg/kg)^{c} |

Vehicle

| | | | | | | | | |
|---|---|---|---|---|---|---|---|---|
| 2116 | 36 | 1 | 2 | 14.83 | 9.49 | 0.26 | 0 | 0 |
| | | 2 | 2 | 14.96 | 9.57 | 0.27 | 0 | 0 |
| | | Cumulative | 4 | 29.79 | 19.06 | 0.53 | 0 | 0 |

sIL-13Rα2-IgG

| | | | | | | | | |
|---|---|---|---|---|---|---|---|---|
| 2119 | 37 | 1 | 2 | 14.90 | 9.54 | 0.26 | 5.24 | 0.14 |
| | | 2 | 2 | 14.99 | 9.59 | 0.26 | 5.28 | 0.14 |
| | | Cumulative | 4 | 29.89 | 19.13 | 0.52 | 10.52 | 0.28 |
| 2121 | 35 | 1 | 2 | 14.97 | 9.58 | 0.27 | 5.27 | 0.15 |
| | | 2 | 2 | 14.95 | 9.57 | 0.27 | 5.26 | 0.15 |
| | | Cumulative | 4 | 29.92 | 19.15 | 0.55 | 10.53 | 0.30 |

Notes:
BP = blister pack.
Assumption: IH delivery efficiency = 64% for both vehicle and sIL-13Rα2-IgG.
Each Active BP contained 55% API
^{a}Nominal Dose (mg) = Actual BP powder fill mass (mg) × #BP
^{b}Estimated Vehicle Lung Dose (mg) = Nominal Dose (mg) × IH delivery efficiency
^{c}Estimated Lung Dose (mg/kg) = Estimated Lung Dose (vehicle or active, mg)/BW (kg)
^{d}Estimated API Lung Dose (mg) = Nominal Dose (mg) × fraction of API in total powder × IH delivery efficiency

Approximately 10 mg of vehicle powder (0.27 mg/kg) was delivered each day to the vehicle control sheep (#2116) for a total of 0.53 mg/kg of powder cumulative dose over two days. For sIL-13Rα2-IgG, the daily powder dose was approximately 10 mg (0.26 or 0.27 mg/kg). The cumulative powder dose was approximately 19 mg (0.52 or 0.55 mg/kg) over two days. The dose of sIL-13Rα2-IgG delivered per day was approximately 5 mg (0.14 or 0.15 mg/kg). The cumulative dose of sIL-13Rα2-IgG delivered over two days was approximately 11 mg (0.28 or 0.30 mg/kg).

6.2 Effect of Vehicle Dose on Asthmatic Response

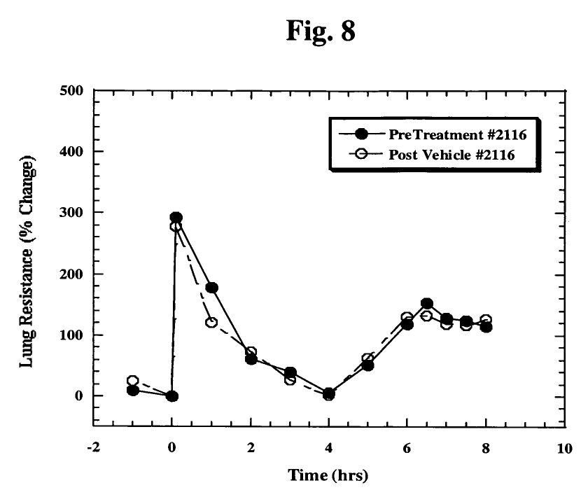

The administration of 2 BP's of vehicle twice prior to antigen challenge had no effect on the asthmatic response in the sheep (see FIG. 8). Treatment with approximately 10 mg of vehicle dry powder at 24 and 2 hrs prior to antigen challenge had no effect on the early (0-3 hrs post antigen) or late (4-8 hrs post antigen) asthmatic response (% change in R_{L} from baseline).

6.3 Effect of sIL-13αR2-IgG on the Sheep Asthmatic Response

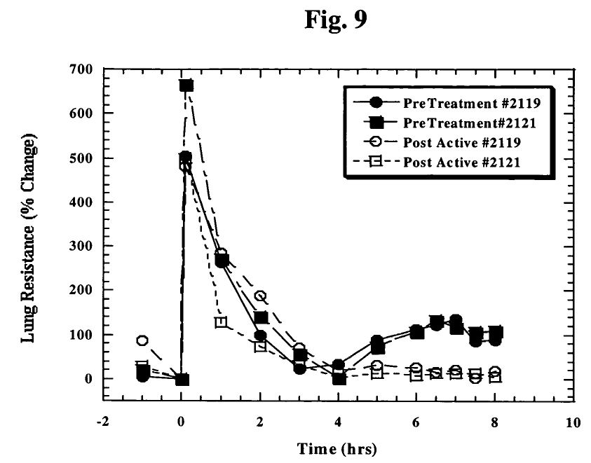

Treatment with sIL-13Rα2-IgG (approximately 5 mg, 0.14 or 0.15 mg/kg) at 24 and 2 hrs prior to antigen challenge did inhibit the late asthmatic response in both sheep (FIG. 9).

### 7. CONCLUSIONS

Treatment with inhaled vehicle dry powder had no effect on the asthmatic response to ascaris sum antigen challenge in the sheep. Treatment with two doses (approximately 5 mg per dose; 0.14 or 0.15 mg/kg per dose) of inhaled sIL-13Rα2-IgG (24 and 2 hrs prior to antigen challenge) inhibited the late phase bronchoconstrictive response to the antigen and BHR in the sheep.

### Example 30, and Comparative Examples 5 and 6

### Preparation and Characterization of sIL-13Rα2-IgG for a Sheep Pulmonary Dosing Study

### 1. SUMMARY

The objectives of these Examples were to (1) prepare dry powders of sIL-13Rα2-IgG and a vehicle powder containing excipients only for an inhalation efficacy sheep study; and (2) to evaluate the aerosol, solid state and chemical stability of the powders over the course of the study.

The sIL-13Rα2-IgG powder was prepared using the same formulation and processing conditions used to prepare powders in the formulation feasibility study (Examples 19-27). The active formulation (formulation A from the feasibility study) contained 55% sIL-13Rα2-IgG with the remainder of the formulation being a mixture of excipients. A vehicle control was formulated that lacked the active pharmaceutical ingredient (API). The resulting powders were assayed for aerosol performance and aggregate content.

There was no evidence of change in aerosol performance or increase in soluble aggregates of the sIL-13Rα2-IgG or the vehicle formulations as a result of shipping to the animal facility or at controlled stability storage (Table 23).

The powders were found to be acceptable and stable during the course of the sheep pulmonary study.

### 2. OBJECTIVE

The objectives of this project were to (1) prepare dry powders of sIL-13Rα2-IgG and a vehicle powder containing excipients only for an inhalation efficacy sheep study; and (2) to evaluate the aerosol, solid state and chemical stability of the powders over the course of the study.

### 3. SCOPE

The sIL-13Rα2-IgG and vehicle control powders were prepared and filled at 7.5 mg into blister packages (BPs) for use in the pulmonary sheep study. The sIL-13Rα2-IgG formulation was originally prepared in the feasibility study as Formulation A. In the feasibility study, the BPs were filled and tested at 5 mg, however in the current study BPs are filled at 7.5 mg to accommodate dosing requirements. Powders were delivered to the sheep using a pneumatically driven aerosol delivery system (PDADS).

The sIL-13Rα2-IgG and vehicle control powders were analyzed for powder delivery using the PDADS before animal dosing. The results were recorded to determine aerosol dosing efficiency.

The aerosol performance, solid-state properties, and chemical stability of the sIL-13Rα2-IgG formulations were evaluated after spray drying (initial time point) and after 3 weeks of storage at several conditions. For the stability studies, powders were filled into BPs, which were then sealed in foiled pouches with desiccant.

### 4. MATERIALS AND METHODS

4.1 Formulation Preparation

The sIL-13Rα2-IgG formulation was prepared by diafiltering sIL-13Rα2-IgG (free of Tween 80) into a 2.5 mM citrate buffer, pH 6.5. Excipients were added to enhance the aerosol performance and chemical stability of the resulting powder. Vehicle-1 powder was prepared by combining the excipients in the proportions found in Table 20 and adjusting pH to 6.5. These formulations were spray dried on a laboratory-scale Büchi system. Vehicle-2 powder was a non-citrate control powder prepared at pH 7.0.

The compositions (weight-by-weight; w/w) of the three formulations are shown in Table 20. For the purpose of this report and for calculating dose and solids content in the formulations, the active pharmaceutical ingredient (API) content shown in Table 20 refers to the proportion of the powder represented by the aglycone sIL-13Rα2-IgG; the glycan percentage is calculated as 14% of the total mass of sIL-13Rα2-IgG.

TABLE 20
Weight percentages of sIL-13Rα2-IgG and vehicle control powders

| | Formulation | API | Glycan from | Sucrose | Trileucine | Leucine | Citrate |
|---|---|---|---|---|---|---|---|
| Example | Designation | (%) | sIL-13Rα2-IgG (%) | (%) | (%) | (%) | Buffer (%) |
| 30 | sIL-13Rα2-IgG | 55 | 9 | 10 | 20 | 0 | 6 |
| Comp. 5 | Vehicle-1 | 0 | 0 | 10 | 20 | 64 | 6 |
| Comp. 6 | Vehicle-2 | 0 | 0 | 0 | 26 | 74 | 0 |

4.2 Formulation Evaluation

To test the stability of the sIL-13Rα2-IgG and Vehicle-1 formulations over 3 weeks, the powders were filled into blister packs (BPs) at 7.5 mg total fill weight. The BPs were then sealed in foil pouches with desiccant. Samples were either shipped from California to Florida for testing or stored in incubation chambers under 25° C./60% RH or 40° C./75% RH. The aerosol and solid state analysis of the packaged powders were performed using the stored BPs, and the chemical tests were performed on reconstituted solutions of the packaged powder (BPs). These analyses were conducted at the initial time and after 3 weeks of storage under the conditions indicated in Table 21.

TABLE 21
Stability protocol for sIL-13Rα2-IgG and Vehicle-1 Formulations

| | | | BPs in Pouch with Desiccant | | |
|---|---|---|---|---|---|
| | | | 3 weeks @ | 3 weeks @ | Shipped from CA to |
| Parameter | Assay | Initial | 25° C./60% RH^{a} | 40° C./75% RH^{a} | FL and returned to CA |
| Aerosol performance | ED | X | X | X | X |
| | MMAD | X | X | X | X |
| Chemical | SEC | X | X | X | X |
| | SDS-PAGE | X | X | X | X |
| | UV | X | X | X | X |
| Residual solvent content | TGA | X | X | X | X |
| Gross morphology | SEM | X | X | X | X |

a-Testing on sIL-13Rα2-IgG formulation only

The specific methods used to characterize the aerosol performance and assess the stability of sIL-13Rα2-IgG are listed in Table 22.

TABLE 22
Methods used to characterize sIL-13Rα2-IgG powder

| | |
|---|---|
| Parameter | Method |

Aerosol Performance Analyses

| | |
|---|---|
| Emitted dose (ED) | Gravimetric analysis, flow |
| | rate = 30.0 L/min (n = 10) |
| Particle size | Gravimetric-based Andersen |
| distribution (PSD): Mass | cascade impaction (ACI) |
| median aerodynamic | (stage cut-off sizes: 9, 5.8, |
| diameter (MMAD), | 4.7, 3.3, 2.1, 1.1, 0.7, and |
| fine particle fraction | 0.4 μm, and filter) at a flow |
| (FPF_{<3.3 μm}), fine | rate of 28.3 L/min (n = 3) |
| particle dose | |
| (FPD_{<3.3 μm}) | |
| PDADS delivery efficiency | Percentage of BP fill weight |
| | emitted from the PDADS after |
| | actuation of one BP. Powder |
| | from a BP was actuated from |
| | a PDS inhaler into a dis- |
| | persion chamber. A pneumatically |
| | driven piston followed by |
| | a bolus of air pushed the |
| | dispersed powder into an |
| | endotracheal tube. The delivery |
| | efficiency to the animal is |
| | the gravimetric fraction of the |
| | powder collected on a filter |
| | connected to the end of the |
| | endotracheal tube, divided by |
| | the actual BP fill weight, and |
| | expressed as a percentage. |
| | The delivery efficiency is used |
| | to calculate the estimated |
| | dose (mg). |

Solid-State Analyses

| | |
|---|---|
| Gross morphology | Scanning electron microscopy |
| | (SEM), Au/Pd sputter coating |

Chemical Analyses

| | |
|---|---|
| Residual solvent content | Thermogravimetric analysis |
| | (TGA) |
| Degradation and | Size-exclusion chromatography |
| aggregation | (SEC): (total soluble) |
| | aggregation |
| | SDS-PAGE: covalent |
| | aggregation and degradation |

4.2.1 Evaluation of Aerosol Performance

The aerosol performance of the sIL-13Rα2-IgG and the vehicle-1 powders were determined using a PDS inhaler, as disclosed in U.S. Pat. No. 6,257,233, which is incorporated by reference herein in its entirety. Aerosol performance was evaluated by gravimetrically determining the percent emitted dose (% ED; the percentage of BP fill weight emitted from the inhaler after the actuation of one BP), and by gravimetrically determining the particle size distribution (PSD) of the formulations filled into the BPs using an Andersen cascade impactor (ACI). PSD parameters included mass median aerodynamic diameter (MMAD), fine particle fraction (FPF_{<3.3 μm}; percentage of delivered particles with aerodynamic diameters less than 3.3 μm), and fine particle dose (FPD_{<3.3 μm}; the mass of aglycone sIL-13Rα2-IgG API delivered in particles <3.3 μm). A summary of the aerosol methods is given in Table 22, above.

From the various historical human clinical studies using both gamma scintigraphy and pharmacokinetics, the amount of sIL-13Rα2-IgG that would be delivered to the lung in humans was estimated as follows for the PDS:
Dose_{lung}=φ_{L}×φ_{ED}×BP_{fill}×Wt_{AI} Equation 1
where φ_{L} is the fraction deposited in the human lung based on historical clinical and preclinical data, φ_{ED} is the emitted dose; BP_{fill} is the fill weight of the BP, and Wt_{AI} is the weight percent active ingredient in the formulation.
4.2.1.1 Powder Characterization on the Pneumatically Driven Aerosol Delivery System

Powder from a BP is actuated from a PDS into a dispersion chamber. A pneumatic driven piston pushes the disperse powder into an intratracheal tube. The aerosol efficiency for each of the powders using the PDADS was determined gravimetrically. The percent emitted dose (% ED) is the percentage of BP fill weight emitted from the PDADS after the actuation of one BP.

4.2.2 Physical and Chemical Assessment

The gross morphology of the particles was assessed by scanning electron microscopy (SEM). The powders were evaluated by size exclusion chromatography (SEC) for total soluble aggregation and by sodium dodecyl sulfate-polyacrylamide gel electrophoresis (SDS-PAGE) for covalent aggregation. Moisture content of the powders was determined by thermogravimetric analysis (TGA), by heating samples to 110° C. at 10° C./min and holding the temperature at 110° C. for 20 minutes.

### 5. RESULTS AND DISCUSSION

After spray drying and packaging (filling into BPs and sealing into foil pouches with desiccant), and after 3 weeks of stability storage under the test conditions, the sIL-13Rα2-IgG and the vehicle-1 formulations did not exhibit any change in aerosol performance or increase chemical degradation.

5.1 Aerosol Performance

The spray-dried powders were packaged and aerosol performance tested at initial time and after 3 weeks of storage. The aerosol performance results of the sIL-13Rα2-IgG formulation and the vehicle-1 formulations are listed in Table 23. No changes in aerosol performance were observed in either the sIL-13Rα2-IgG or the vehicle control, under any of the stability conditions tested.

TABLE 23
Aerosol performance of sIL-13Rα2-IgG and vehicle-1 formulations immediately after
spray drying (initial) and after 3 weeks of storage under indicated conditions

| | | | | PSD Data | | | |
|---|---|---|---|---|---|---|---|
| | | % ED @ 30 L/min | | MMAD | FPF_{<3.3 μm} | FPM_{<3.3 μm} | FPD_{<3.3 μm} |
| | Stability | Mean | | (μm) | (%) | (mg) | (mg) |
| Formulation | Conditions | (n = 10) | RSD | (n = 3) | (n = 3) | (n = 3) | (n = 3) |
| Target | n/a | >60 | — | <3.5 | >45 | — | — |
| sIL-13Rα2-IgG | Initial | 83 | 5 | 3.1 | 54 | 5.7 | 3.1 |
| | 25° C./60% RH/ | 82 | 2 | 3.1 | 54 | 5.9 | 3.3 |
| | desiccated | | | | | | |
| | 40° C./75% RH/ | 78 | 4 | 3.2 | 55 | 5.8 | 3.2 |
| | desiccated | | | | | | |
| | Shipped from CA | 79 | 3 | 3.2 | 53 | 5.8 | 3.2 |
| | to FL and returned | | | | | | |
| | to CA-desiccated | | | | | | |
| Vehicle-1 | Initial | 83 | 3 | 3.0 | 55 | 5.8 | na |
| | Shipped from CA | 82 | 3 | 3.1 | 54 | 5.7 | na |
| | to FL and returned | | | | | | |
| | to CA-desiccated | | | | | | |

RSD = relative standard deviation.
na = not applicable

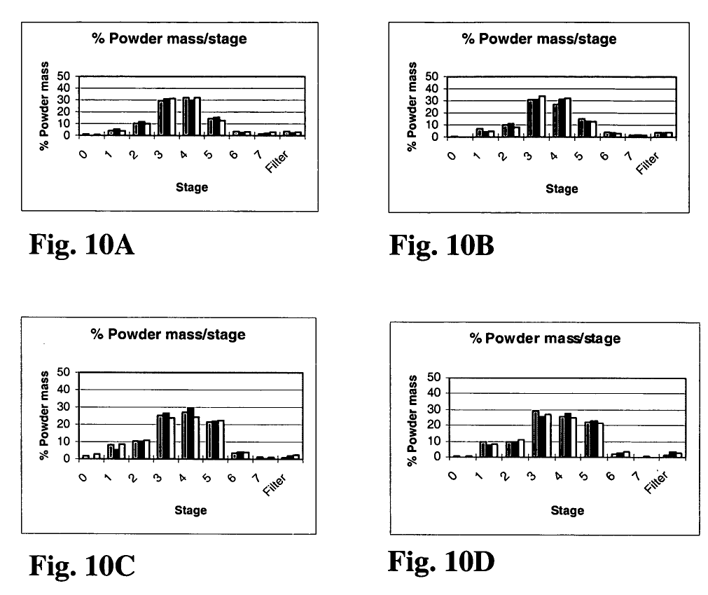

The particle size distribution profiles of the powders are shown in FIGS. 10A-10D, wherein the particle size distribution profiles were determined using the particle size cutoffs shown in Table 24. FIG. 10A shows the initial particle distribution profile for formulation A, and FIG. 10B shows the particle distribution profile for formulation A after shipment from California to Florida and back to California in BPs stored in foil pouches and desiccated. FIG. 10C shows the initial particle distribution profile for vehicle 1, and FIG. 10D shows the particle distribution profile for vehicle 1 after shipment from California to Florida and back to California in BPs stored in foil pouches and desiccated.

TABLE 24
Particle Size Cutoff

| | Particle Size Cutoff |
|---|---|
| Stage | (μm) |
| 0 | 9.0 |
| 1 | 5.8 |
| 2 | 4.7 |
| 3 | 3.3 |
| 4 | 2.1 |
| 5 | 1.1 |
| 6 | 0.7 |
| 7 | 0.4 |
| Filter | <0.4 |

The predicted lung deposition of the test sIL-13Rα2-IgG formulation calculated according to Equation 1, based on actuation of one BP with a fill weight of 7.5 mg was 1.9 mg.

Powder Delivery Efficiency Using PDADS

The spray-dried sIL-13Rα2-IgG and the vehicle-1 control powder were analyzed for powder delivery efficiency using the PDADS. In California, the PDADS was connected to an air line that pushed the aerosolized powder through the system. The emitted dose values are recorded in Table 25. However for planning purposes, 50% emitted dose was used to estimate the dose. This number was used to determine the number of blister packs to fill.

TABLE 25
Powder delivery efficiency using PDADS with chase air tested

% ED (n = 10)

| Formulation | Mean | RSD |
|---|---|---|
| sIL-13Rα2α2-IgG | 42 | 11 |
| Vehicle-1 | 45 | 21 |

Key to abbreviations:
ED = emitted dose,
RSD = relative standard deviation.

The sIL-13Rα-IgG and both vehicle control powders were analyzed for powder delivery using the PDADS before animal dosing. The results were recorded to determine aerosol dosing efficiency (Table 26). These values were used to calculate the dose delivered to the sheep.

The delivery efficiencies of the spray-dried powders are higher when tested in Florida compared to the same powder tested in California. The increase in efficiency is likely attributed to the difference between setups: the one in Florida using the ventilator connected to the PDADS, versus the other in California using chase air. Since the sheep were dosed in Florida with the ventilator, the efficiencies measured with the ventilator were used to determine dose.

TABLE 26
Powder dosing efficiency using PDADS in Florida

% ED

| | | Mean | |
|---|---|---|---|
| Example | Formulation | (n = 5) | RSD |
| 30 | sIL-13Rα2-IgG | 63 | 3 |
| | | 66 | 3 |
| Comp. Ex. 1 | Vehicle-1 | 63 | 3 |
| | | 62 | 2 |
| | | 69 | 2 |
| Comp. Ex. 2 | Vehicle-2 | 62 | 1 |

5.2 Morphology

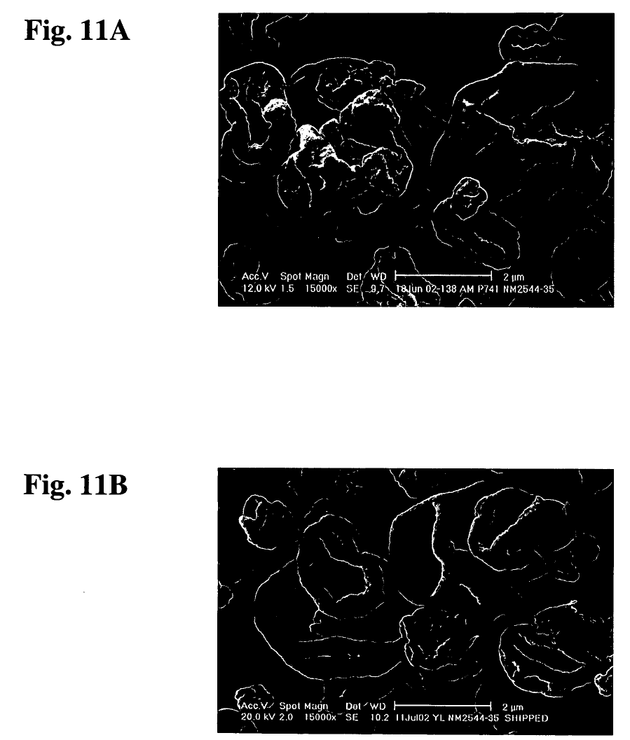

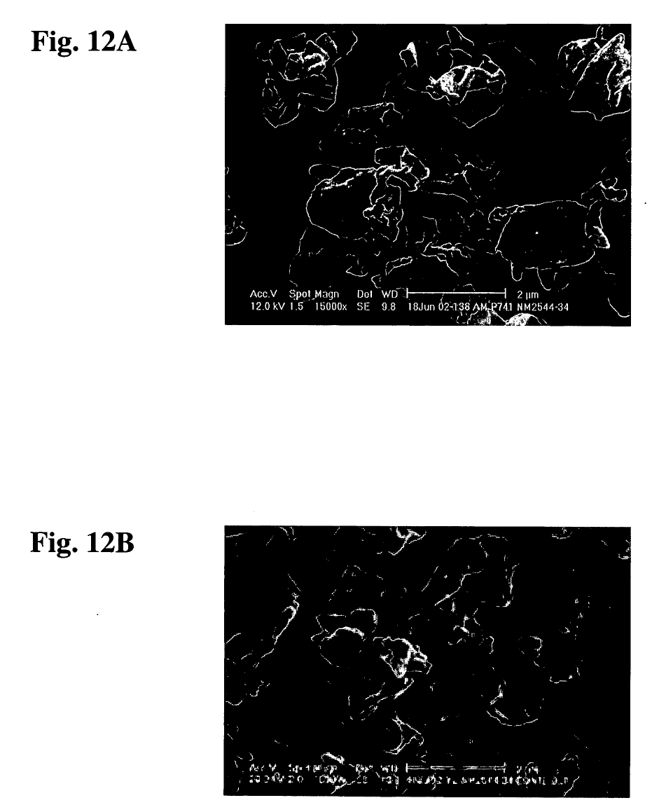

FIGS. 11A and 11B are SEM images of the sIL-13Rα2-IgG formulation before and after shipment from California to Florida and back to California in BPs stored in foil pouches with desiccant, respectively. FIGS. 12A and 12B are SEM images of the vehicle-1 formulation before and after shipment from California to Florida and back to California in BPs stored in foil pouches with desiccant, respectively. There were no visible changes in gross morphology to either the samples that were shipped from California to Florida and returned to California or to any of the test powders after 3 weeks of storage at 25° C./60% RH or 40° C./75% RH.

5.3 Chemical Stability

5.3.1 Residual Solvent Content

Table 27 shows the residual solvent content of each formulation after spray drying and after 3 weeks of storage in BPs sealed in foil pouches with desiccant. sIL-13Rα2-IgG BPs that were shipped from California to Florida and returned to California for analysis increased from 3.4% at initial to 3.7%. As TGA is used to estimate moisture, this change is viewed as being within the error of the assay.

TABLE 27
Residual solvent content (%) of sIL-13Rα2-IgG
and vehicle control formulations

| | | BPs in pouch with desiccant | | |
|---|---|---|---|---|
| | | | | Shipped from |
| | | 3 weeks | 3 weeks | CA to FL and |
| | | @ 25° C./ | @ 40° C./ | returned |
| Formulation | Initial | 60% RH | 75% RH | to CA |
| sIL-13Rα2- | 3.4 | 3.0 | 3.0 | 3.7 |
| IgG | | | | |
| Vehicle-1 | 1.6 | na | na | 1.5 |

5.3.2 Aggregation by SEC

SEC analyses were performed on powders that had been filled into BPs and sealed in foil pouches with desiccant. The sIL-13Rα2-IgG powders were reconstituted in water and analyzed. The sIL-13Rα2-IgG size-exclusion chromatograms indicated no increase in soluble aggregate formation in either the packaged samples shipped form California to Florida and returned to California or the samples stored at 25° C./60% RH or at 40° C./75% RH.

5.3.3 Aggregation by SDS-PAGE

SDS-PAGE showed no evidence of sIL-13Rα2-IgG degradation relative to the API, or of covalent aggregate formation, in either the packaged samples shipped from California to Florida and returned to California or the samples stored at 25° C./60% RH or at 40° C./75% RH.

### 6. CONCLUSIONS

There was no evidence of change in aerosol performance or increase in soluble aggregates of the sIL-13Rα2-IgG or the vehicle formulations as a result of shipping to the animal facility or at controlled stability storage (Table 23).

The powders were found to be acceptable and stable during the course of the sheep pulmonary study.

### Example 31

### Preparation and Characterization of Spray-dried sIL-13Rα2-IgG for Sheep Pulmonary Dosing Study

### 1. SUMMARY

A soluble interleukin-13 receptor (sIL-13Rα2-IgG) formulation powder was manufactured for a sheep pulmonary delivery study. The spray-dried sIL-13Rα2-IgG powder, either shipped to the animal facility, or stored at 25° C./60% RH for 11 weeks, was found to have acceptable performance and stability at the beginning and end of the animal study.

The sIL-13Rα2-IgG powder was analyzed to estimate the delivered dose using a pneumatically driven aerosol delivery system (PDADS) before dosing animals. The results indicated that the aerosol delivery was acceptable for the goals of the animal study.

### 2. OBJECTIVE

The objectives of this project were: to prepare spray-dried sIL-13Rα2-IgG powder for an inhalation efficacy study in sheep, and to evaluate the aerosol performance and physicochemical stability of the powder at the beginning and end of the study.

In Examples 28 and 29, sIL-13Rα2-IgG was dosed 24 hrs and again at 2 hrs prior to antigen challenge (0.15 mg/kg per dose) in a sheep asthma model and was shown to be efficacious. The current study was designed to determine if a single treatment of sIL-13Rα2-IgG given at 24 hours prior to antigen challenge would be efficacious in the sheep model. Two doses, 0.07 mg/kg and 0.14 mg/kg were tested in this study.

### 3. METHODS

3.1 Active Pharmaceutical Ingredient (API)

The approximate molecular weight of sIL-13Rα2-IgG (free of Tween 80) is 142 kDa. The protein is glycosylated, and the glycone portion constitutes 14% of the total mass of the sIL-13Rα2-IgG. The extinction coefficient used to determine the protein concentration was 2.18 mL mg⁻¹ cm⁻¹ at 280 nm, and was not adjusted for the effects of glycosylation.

3.1.1 Formulation Preparation

The sIL-13Rα2-IgG formulation was prepared by diafiltering the sIL-13Rα2-IgG into a 2.5 mM citrate buffer at pH 6.5, and excipients were added to produce aerosolizable particles and preserve physiochemical stability of the resulting powder. The sIL-13Rα2-IgG formulation previously was referred to as Formulation A in the feasibility study (see, e.g., Examples 19-27). Powder was spray dried on a laboratory-scale Büchi system. The formulation composition of the powder is summarized in Table 28.

The API content shown in Table 28 refers to the proportion of the powder represented by the aglycone (non-carbohydrate) part of sIL-13Rα2-IgG; the glycone percentage is calculated as 14% of the total mass of sIL-13Rα2-IgG.

TABLE 28
Weight percentages of spray-dried sIL-13Rα2-IgG

| | | Glycone from | | | |
|---|---|---|---|---|---|
| Formulation | API | sIL-13Rα2-IgG | Sucrose | Trileucine | Citrate |
| Designation | (%) | (%) | (%) | (%) | Buffer (%) |
| sIL-13Rα2-IgG | 55 | 9 | 10 | 20 | 6 |

3.1.2 BP Filling, Packaging, and Storage

The spray-dried sIL-13Rα2-IgG was manually filled into blister packs (BPs) at a nominal fill weight of 7.50 mg/BP. The BPs (10 BPs/plastic BP holder) were then sealed in a foil pouch (one holder/pouch) with desiccant. Samples were either placed into a cardboard box and shipped from California to Florida for animal dosing, or stored in California in chambers maintained at 25° C./60% RH or 40° C./75% RH.

3.1.3 Analytical Procedures

The following parameters were assessed for the spray-dried sIL-13Rα2-IgG powders using the indicated analytical techniques.
Aerosol performance was assessed using a PDS inhaler.
   Emitted dose (ED): the percentage of the BP contents emitted from the inhaler after actuation. The gravimetric analysis was performed at a flow rate of 30.0 L/min.
   Particle size distribution (PSD) parameters included mass median aerodynamic diameter (MMAD), fine particle mass (FPM_{<3.3 μm}; cumulative weight (mg) of delivered particles with aerodynamic diameters <3.3 μm), and fine particle dose (FPD_{<3.3 μm}; the mass of aglycone part of sIL-13Rα2-IgG delivered in particles with aerodynamic diameters <3.3 μm). FPD_{<3.3 μm} was calculated by multiplying FPM_{<3.3 μm} by the nominal dose fraction. PSD parameters were determined by gravimetric-based Andersen cascade impaction (ACI) (stage cut-off sizes: 9.0, 5.8, 4.7, 3.3, 2.1, 1.1, 0.7, and 0.4 μm, and filter) at a flow rate of 28.3 L/min with PDS inhaler.
   Pneumatically dosing aerosol delivery system (PDADS) delivery efficiency: percentage of powder emitted from the PDADS onto a filter after actuation. Powder from a BP was actuated from a PDS inhaler into a dispersion chamber. A pneumatically driven piston followed by a bolus of air pushed the dispersed powder into an endotracheal tube. The delivery efficiency to the animal is the gravimetric mass of the powder collected on a filter connected to the end of the endotracheal tube, divided by the actual BP fill weight, and expressed as a percentage. The delivery efficiency is used to calculate the estimated dose (mg).
Physiochemical analysis
   Gross morphology was determined by scanning electron microscopy (SEM) using Au/Pd sputter coating.
   Residual solvent content was determined by thermogravimetric moisture analysis (TGA).
   Total soluble aggregation was determined by size exclusion chromatography (SEC) and covalent aggregation and degradation were determined by sodium dodecyl sulfate-polyacrylamide gel electrophoresis (SDS-PAGE).
      3.1.4 Formulation Evaluation

The spray-dried sIL-13Rα2-IgG powder was analyzed at the initial time point, then stored in California or shipped from California to Florida. At the conclusion of the animal studies, samples of the spray-dried sIL-13Rα2-IgG powder used to dose the animals were returned from Florida to California and analyzed along with the stored sIL-13Rα2-IgG powder retained in California and stored in incubation chambers.

Stability analyses were conducted on the spray-dried sIL-13Rα2-IgG powder as listed in Table 29.

TABLE 29
Stability protocol for spray-dried sIL-13Ra2-IgG powder

| | | | BPs in Pouch with Desiccant, Week 11 | | |
|---|---|---|---|---|---|
| | | | | | Shipped from CA |
| | | | | | to FL and returned |
| Parameter | Assay | Initial | 25° C./60% RH | 40° C./75% RH | to CA |
| Aerosol | ED | X | X | X | X |
| Performance | determination | | | | |
| | Aerosol | X | X | X | X |
| | particle size | | | | |
| | determination | | | | |
| Physiochemical | SEC | X | X | X | X |
| | SDS-PAGE | X | X | X | X |
| | TGA | X | X | X | X |
| | SEM | X | X | X | X |

Key to abbreviations:
ED = emitted dose,
SEC = size-exclusion chromatography,
SDS-PAGE = sodium dodecyl sulfate-polyacrylamide gel electrophoresis,
TGA = thermogravimetric analysis,
SEM = scanning electron microscopy.

### 4. RESULTS AND DISCUSSION

4.1 Aerosol Performance

4.1.1 Performance Using the PDS

The spray-dried powder was packaged and aerosol performance was tested at the initial time and after 11 weeks of storage. The aerosol performance of the spray-dried sIL-13Rα2-IgG powder was acceptable under all the testing conditions and at the end of the animal studies, as shown in Table 30.

TABLE 30
Aerosol performance of spray-dried sIL-13Rα2-IgG powders under indicated conditions

| | | | | Aerosol PSD | | |
|---|---|---|---|---|---|---|
| | | | | (n = 1) | | |
| | | % ED @ 30 L/min | | | FPM_{<3.3 μm} | FPD_{<3.3 μm} |
| | Stability | (n = 5) | | MMAD | (mg) | (mg) |
| Formulation | Conditions | Mean | RSD (%) | (μm) | Per BP | Per BP |
| sIL-13Rα2-IgG | Initial | 83^{c} | 2 | 3.2^{a} | 2.9^{a} | 1.6^{a} |
| | 25° C./60% RH | 84 | 2 | 3.3^{b} | 2.7^{b} | 1.5^{b} |
| | desiccated | | | | | |
| | 40° C./75% RH | 81 | 2 | 3.4^{b} | 2.5^{b} | 1.4^{b} |
| | desiccated | | | | | |
| | Shipped from CA to | 86 | 2 | 3.4^{b} | 2.5^{b} | 1.4^{b} |
| | FL and returned to | | | | | |
| | CA/desiccated | | | | | |

Key to abbreviations:
BP= Blister Pack,
ED = emitted dose,
FPD = fine particle dose,
FPM = fine particle mass,
MMAD = mass median aerodynamic diameter,
PSD = particle size distribution,
RSD = relative standard deviation.
^{a}Aerosol PSD testing with three BPs
^{b}Aerosol PSD testing with two BPs
^{c}ED testing with n = 10

4.1.2 Powder Delivery Efficiency Using PDADS

The spray-dried sIL-13Rα2-IgG powder was analyzed for powder delivery efficiency using the PDADS. In California, the PDADS was connected to an air line that pushed the aerosolized powder through the system. The PDADS emitted dose values are recorded in Table 31.

TABLE 31
Powder delivery efficiency using PDADS with chase air tested

| | |
|---|---|
| Testing | % ED (n = 5) |

| Formulation | Date | Mean | RSD (%) |
|---|---|---|---|
| sIL-13Rα2-IgG | Month 0 | 46 | 6 |
| | Month 13 | 46 | 18 |

Key to abbreviations:
ED = emitted dose,
RSD = relative standard deviation.

In Florida, the spray-dried sIL-13Rα2-IgG powder was analyzed for powder delivery efficiency using the PDADS just before animal dosing. The PDADS was connected to a ventilator that pushed the aerosolized powder through the system. The experimental results are recorded in Table 32.

The delivery efficiencies of the spray-dried powders are higher when tested in Florida compared to the same powder tested in California. The increase in efficiency is likely attributed to the difference between setups: the PDADS set up in Florida was connected to a ventilator whereas the PDADS set up in California was connected to a chase air set-up. Since the sheep were dosed in Florida with the ventilator, the efficiencies measured with the ventilator were used to calculate the estimated dose to the sheep.

TABLE 32
Powder delivery efficiency using PDADS
with ventilator tested in Florida

| | | % ED (n = 5) | | % ED | |
|---|---|---|---|---|---|
| | Dosing | Daily | | Overall | Overall RSD |
| Formulation | Date | Mean | RSD (%) | Mean | (%) |
| sIL-13Rα2-IgG | Day 0 | 63 | 3 | 64 | 4 |
| | Day 2 | 65 | 5 | | |

Key to abbreviations:
ED = emitted dose,
RSD = relative standard deviation.

4.2 Physiochemical Stability
4.2.1 Morphology

There were no visible differences in gross morphology to either the sIL-13Rα2-IgG powder tested before the study, to powder that was shipped from California to Florida and returned to California or to the sIL-13Rα2-IgG powders stored for 11 weeks at 25° C./60% RH and 40° C./75% RH.

4.2.2 Residual Solvent Content

Table 33 shows the estimated residual solvent content of each formulation determined using TGA after spray drying and after 11 weeks of storage in BPs sealed in foil pouches with desiccant. The moisture content in the sIL-13Rα2-IgG BPs that were shipped from California to Florida and returned to California for analysis increased from an initial value of 1.8% to 2.7%. This change is within the error of the TGA assay, which is used to estimate moisture.

TABLE 33
Residual solvent content (%) of sIL-13Rα2-IgG formulation

| | | BPs in pouch with desiccant | | |
|---|---|---|---|---|
| | | | | Shipped from |
| | | 11 weeks | 11 weeks | CA to FL and |
| | | @ 25° C./ | @ 40° C./ | returned |
| Formulation | Initial | 60% RH | 75% RH | to CA |
| sIL-13Rα2- | 1.8 | 2.4 | 2.3 | 2.7 |
| IgG | | | | |

Key to abbreviations:
BP = blister pack,
RH = relative humidity.

4.2.3 Aggregation by SEC

The SEC analyses were performed on powders that had been filled into BPs and sealed in foil pouches with desiccant. The spray-dried sIL-13Rα2-IgG powder was reconstituted in water and analyzed.

The HMW aggregate content of the formulated pre-spray dry solution increased by about 3.5% relative to the drug substance. The increase in HMW aggregate of the formulated solution is likely attributed to the diafiltration process of the drug substance. In previous studies, the spray-dried powder with the same formulation composition analyzed at initial time point did not show a significant increase (0.3% and 0%) in HMW content when compared to the drug substance. In the current study as well as in previous studies, the spray-drying process did not appreciably change the HMW content.

The sIL-13Rα2-IgG size-exclusion chromatograms showed an increase of up to 3.1% HMW aggregate, relative to a change from initial in the stability samples stored at 40° C./75% RH. Samples that were stored at 25° C./60% RH or shipped from California to Florida and returned to California did not change for the duration of the study.

4.2.4 Aggregation Measured by SDS-PAGE

There was no evidence of sIL-13Rα2-IgG degradation relative to the API, or of covalent aggregate formation, in either the packaged samples shipped from California to Florida and returned to California or the samples stored at 25° C./60% RH or at 40° C./75% RH.

### 5. CONCLUSIONS

A soluble interleukin-13 receptor (sIL-13Rα2-IgG) formulation powder was manufactured for a sheep pulmonary delivery study. The spray-dried sIL-13Rα2-IgG powder, either shipped to the animal facility, or stored at 25° C./60% RH for 11 weeks, was found to have acceptable performance and stability.

The sIL-13Rα2-IgG powder was analyzed to estimate the delivered dose using a pneumatically driven aerosol delivery system (PDADS) before dosing animals. The results indicated that the aerosol delivery was acceptable for the goals of the animal study.

### Example 32

### Pharmacokinetics and Pharmacodynamics of sIL-13Rα2-IgG at 0.14 and 0.7 mg/kg in Sheep After Inhalation

Sheep were treated with either a single inhalation (1 blister pack, BP); target dose=0.07 mg/kg) or two inhalations (2 BPs; target dose=0.14 mg/kg) of a dry powder formulation of sIL-13α2-IgG to evaluate efficacy in an antigen challenge model of asthma. The formulation and delivery procedure of this Example were similar to that of Examples 28 and 29.

The dry powder dose was given once at 24 hours prior to antigen challenge. The total amount of dry powder delivered was approximately 5 mg for the single inhalation (1 BP; average 0.17 mg/kg) and 10 mg total for the two inhalations (2 BPs; average 0.27 mg/kg). The total amount of sIL-13Rα2-IgG delivered was approximately 3 mg (1 BP; average 0.10 mg/kg) in one inhalation and approximately 5 mg (2 BPs; average 0.15 mg/kg) in two inhalations. The change in lung resistance was measured over the next 8 hours and 24 hours post-antigen challenge, the nonspecific bronchial hyperresponsiveness (BHR) to carbachol was measured and compared to historic control. A single inhalation (average 0.10 mg/kg in 1 BP) of inhaled sIL-13Rα2-IgG administered 24 hours prior to antigen challenge had no effect on the BHR or the asthmatic response. Delivery of sIL-13Rα2-IgG in two inhalations (average 0.15 mg/kg in 2 BPs) to the sheep once at 24 hours prior to antigen challenge inhibited the late asthmatic response.

The foregoing embodiments and advantages are merely exemplary and are not to be construed as limiting the present invention. The description of the present invention is intended to be illustrative, and not to limit the scope of the claims. Many alternatives, modifications, and variations will be apparent to those skilled in the art.

Having now fully described this invention, it will be understood to those of ordinary skill in the art that the methods of the present invention can be carried out with a wide and equivalent range of conditions, formulations, and other parameters without departing from the scope of the invention or any embodiments thereof.

1. A powder comprising IL-13 antagonist, wherein the powder has a mass median aerodynamic diameter (MMAD) of less than about 10 μm.

2. The powder of claim 1, wherein the IL-13 antagonist comprises at least one IL-13 binding member selected from IL-13Rα1, IL-13Rα2, antibody to IL-13, fragments thereof, homologs thereof, and conjugates thereof.

3. The powder of claim 1, wherein the IL-13 antagonist comprises at least one member selected from IL-13Rα2 and IL-13Rα2-IgG fusion protein.

4. The powder of claim 1, wherein the IL-13 antagonist is present in an amount ranging from about 2 wt % to 100 wt %, based on total weight of the powder.

5. The powder of claim 1, wherein the IL-13 antagonist is present in an amount ranging from about 5 wt % to about 60 wt %, based on total weight of the powder.

6. The powder of claim 1, wherein the MMAD ranges from about 0.5 μm to about 4 μm.

7. The powder of claim 1, wherein the powder has a fine particle fraction (FPF_{<3.3 μm}) ranging from about 0.4 to about 0.95.

8. The powder of claim 1, wherein the powder has a fine particle fraction (FPF_{<4.7 μm}) ranging from about 0.5 to about 0.7.

9. The powder of claim 1, further comprising a pharmaceutically acceptable excipient.

10. The powder of claim 9, wherein the pharmaceutically acceptable excipient comprises at least one member selected from carbohydrate, amino acid, peptide, and buffer.

11. The powder of claim 10, wherein the pharmaceutically acceptable excipient comprises carbohydrate.

12. The powder of claim 11, wherein the carbohydrate comprises at least one member selected from cellobiose, dextrans, dextrose, fructose, galactose, glucitol, glucose, lactitol, lactose, maltodextrans, maltose, mannitol, mannose, melezitose, myoinositol, pyranosyl, raffinose, sorbitol, sorbose, starches, sucrose, trehalose, and xylitol.

13. The powder of claim 11, wherein the carbohydrate comprises at least one member selected from sucrose and mannitol.

14. The powder of claim 10, wherein the pharmaceutically acceptable excipient comprises amino acid.

15. The powder of claim 10, wherein the pharmaceutically acceptable excipient comprises peptide.

16. The powder of claim 15, wherein the peptide comprises at least one member selected from dileucine, leu-leu-gly, leu-leu-ala, leu-leu-val, leu-leu-leu, leu-leu-ile, leu-leu-met, leu-leu-pro, leu-leu-phe, leu-leu-trp, leu-leu-ser, leu-leu-thr, leu-leu-cys, leu-leu-tyr, leu-leu-asp, leu-leu-glu, leu-leu-lys, leu-leu-arg, leu-leu-his, leu-leu-nor, leu-gly-leu, leu-ala-leu, leu-val-leu, leu-ile-leu, leu-met-leu, leu-pro-leu, leu-phe-leu, leu-trp-leu, leu-ser-leu, leu-thr-leu, leu-cys-leu, leu-try-leu, leu-asp-leu, leu-glu-leu, leu-lys-leu, leu-arg-leu, leu-his-leu, and leu-nor-leu.

17. The powder of claim 16, wherein the peptide comprises at least one member selected from dileucine and trileucine.

18. The powder of claim 10, wherein the pharmaceutically acceptable excipient comprises buffer.

19. The powder of claim 18, wherein the buffer comprises at least one member selected from sodium citrate, phosphate, and citric acid.

20. The powder of claim 1, further comprising at least one additional member selected from immune modulating cytokine and cytokine antagonist.

21. The powder of claim 20, wherein the at least one additional member comprises IL-4 antagonist.

22. The powder of claim 1, wherein storage of the powder at 40° C. and 75% relative humidity for one month results in an increase in soluble aggregation of less than 2.5%, as measured by size exclusion chromatography.

23. The powder of claim 1, wherein storage of the powder at 40° C. and 75% relative humidity for one month results in an increase in covalent aggregation of less than 2.5%, as measured by SDS-PAGE.

24. A composition, comprising spray-dried particle comprising IL-13 antagonist.

25. The composition of claim 24, wherein the IL-13 antagonist comprises at least one IL-13 binding member selected from IL-13Rα1, IL-13Rα2, antibody to IL-13, fragments thereof, homologs thereof, and conjugates thereof.

26. The composition of claim 24, wherein the IL-13 antagonist comprises at least one member selected from IL-13Rα2 and IL-13Rα2-IgG fusion protein.

27. The composition of claim 24, wherein the IL-13 antagonist is present in an amount ranging from about 2 wt % to 100 wt %, based on total weight of the spray-dried particle.

28. The composition of claim 24, wherein the IL-13 antagonist is present in an amount ranging from about 5 wt % to about 60 wt %, based on total weight of the spray-dried particle.

29. The composition of claim 24, wherein the composition comprises a powder having a mass median aerodynamic diameter (MMAD) ranging from about 0.5 μm to about 5 μm.

30. The composition of claim 24, wherein the composition comprises a powder having a fine particle fraction (FPF_{<3.3 μm}) ranging from about 0.4 to about 0.95.

31. The composition of claim 24, wherein the composition comprises a powder having a fine particle fraction (FPF_{<4.7 μm}) ranging from about 0.5 to about 0.7.

32. The composition of claim 24, further comprising a pharmaceutically acceptable excipient.

33. The composition of claim 32, wherein the pharmaceutically acceptable excipient comprises at least one member selected from carbohydrate, amino acid, peptide, and buffer.

34. The composition of claim 33, wherein the pharmaceutically acceptable excipient comprises carbohydrate.

35. The composition of claim 34, wherein the carbohydrate comprises at least one member selected from cellobiose, dextrans, dextrose, fructose, galactose, glucitol, glucose, lactitol, lactose, maltodextrans, maltose, mannitol, mannose, melezitose, myoinositol, pyranosyl, raffinose, sorbitol, sorbose, starches, sucrose, trehalose, and xylitol.

36. The composition of claim 34, wherein the carbohydrate comprises at least one member selected from sucrose and mannitol.

37. The composition of claim 33, wherein the pharmaceutically acceptable excipient comprises amino acid.

38. The composition of claim 33, wherein the pharmaceutically acceptable excipient comprises peptide.

39. The composition of claim 38, wherein the peptide comprises at least one member selected from dileucine, leu-leu-gly, leu-leu-ala, leu-leu-val, leu-leu-leu, leu-leu-ile, leu-leu-met, leu-leu-pro, leu-leu-phe, leu-leu-trp, leu-leu-ser, leu-leu-thr, leu-leu-cys, leu-leu-tyr, leu-leu-asp, leu-leu-glu, leu-leu-lys, leu-leu-arg, leu-leu-his, leu-leu-nor, leu-gly-leu, leu-ala-leu, leu-val-leu, leu-ile-leu, leu-met-leu, leu-pro-leu, leu-phe-leu, leu-trp-leu, leu-ser-leu, leu-thr-leu, leu-cys-leu, leu-try-leu, leu-asp-leu, leu-glu-leu, leu-lys-leu, leu-arg-leu, leu-his-leu, and leu-nor-leu.

40. The composition of claim 39, wherein the peptide comprises at least one member selected from dileucine and trileucine.

41. The composition of claim 33, wherein the pharmaceutically acceptable excipient comprises buffer.

42. The composition of claim 41, wherein the buffer comprises at least one member selected from sodium citrate, phosphate, and citric acid.

43. The composition of claim 24, further comprising at least one additional member selected from immune modulating cytokine and cytokine antagonist.

44. The composition of claim 43, wherein the at least one additional member comprises IL-4 antagonist.

45. The composition of claim 24, wherein storage of the powder at 40° C. and 75% relative humidity for one month results in an increase in soluble aggregation of less than 2.5%, as measured by size exclusion chromatography.

46. The composition of claim 24, wherein storage of the powder at 40° C. and 75% relative humidity for one month results in an increase in covalent aggregation of less than 2.5%, as measured by SDS-PAGE.

47. A method of administering IL-13 antagonist to the lungs of a subject, comprising:
dispersing a dry powder composition comprising IL-13 antagonist to form an aerosol, wherein the dry powder composition has a mass median aerodynamic diameter (MMAD) of less than about 10 μm; and
delivering the aerosol to the lungs of the subject by inhalation of the aerosol by the subject, thereby ensuring delivery of the IL-13 antagonist to the lungs of the subject.

48. The method of claim 47, wherein the composition comprises a therapeutically effective amount of the IL-13 antagonist.

49. The method of claim 47, wherein the composition comprises IL-13 antagonist in an amount ranging from about 0.1 mg to about 30 mg.

50. The method of claim 47, wherein the method is repeated so that a therapeutically effective amount of the IL-13 antagonist is delivered to the lungs of the subject.

51. The method of claim 47, wherein the IL-13 antagonist comprises at least one IL-13 binding member selected from IL-13Rα1, IL-13Rα2, antibody to IL-13, fragments thereof, homologs thereof, and conjugates thereof.

52. The method of claim 47, wherein the IL-13 antagonist comprises at least one member selected from IL-13Rα2 and IL-13Rα2-IgG fusion protein.

53. The method of claim 47, wherein the composition comprises spray-dried powder.

54. The method of claim 47, wherein the composition is delivered via a dry powder inhaler.

55. The method of claim 47, wherein the composition is delivered via a metered-dose inhaler.

56. A method of treating an IL-13-related condition, comprising:
pulmonarily administering a therapeutically effective amount of a dry powder comprising IL-13 antagonist, wherein the dry powder a mass median aerodynamic diameter (MMAD) of less than about 10 μm.

57. The method of claim 56, wherein the IL-related condition comprises at least one condition selected from inflammation, asthma, allergies, fibrosis, graft rejection, granuloma, sclerosis, progressive systemic sclerosis, and schistosomiasis.

58. The method of claim 56, wherein the at least one condition comprises idiopathic pulmonary fibrosis, chronic graft rejection, bleomycin-induced pulmonary fibrosis, radiation-induced pulmonary fibrosis, pulmonary granuloma, progressive systemic sclerosis, schistosomiasis, and hepatic fibrosis.

59. The method of claim 56, wherein the therapeutically effective amount ranges from about 0.05 mg/kg to about 5 mg/kg.

60. A method of preparing IL-13 antagonist-containing powder, comprising:
combining IL-13 antagonist, optional excipient, and solvent to form a mixture or solution; and
spray drying the mixture or solution to obtain the powder.

61. The method of claim 60, wherein the powder is dry.

62. The method of claim 60, wherein the powder is suitable for pulmonary administration.
